(19) United States
(12) Patent Application Publication
(10) Pub. No.: US 2025/0034617 A1
(43) Pub. Date: Jan. 30, 2025

(54) RIBOSOME PROFILING VIA ISOTACHOPHORESIS

(71) Applicants: BOARD OF REGENTS, THE UNIVERSITY OF TEXAS SYSTEM, Austin, TX (US); SAN JOSE STATE UNIVERSITY RESEARCH FOUNDATION, San Jose, CA (US)

(72) Inventors: Can CENIK, Austin, TX (US); Crystal M. HAN, Palo Alto, CA (US)

(21) Appl. No.: 18/716,820

(22) PCT Filed: Dec. 6, 2022
(86) PCT No.: PCT/US2022/080982

§ 371 Date: Jun. 5, 2024

Related U.S. Application Data
(60) Provisional application No. 63/286,531, filed on Dec. 6, 2021.

(51) Int. Cl. C12Q 1/6806; C12Q 1/6869

(52) U.S. Cl. CPC C12Q 1/6806; C12Q 1/6869

(57) ABSTRACT

The present disclosure provides methods for profiling the nucleic acids bound to the ribosome by using electrophoresis to separate the nucleic acids bound to the ribosome from a cell lysate. Also, provided herein are devices that may be used to separate these nucleic acids from other parts of the cell and may be used in these methods.

This application claims the benefit of priority to U.S. Provisional Application No. 63/286,531, filed on Dec. 6, 2021, the entire contents of which are hereby incorporated by reference.

### BACKGROUND

This invention was made with government support under Grant no. CA204522 awarded by the National Institutes of Health. The government has certain rights in the invention.

### REFERENCE TO A SEQUENCE LISTING

The sequence listing that is contained in the file named “UTFB.P1298WO.xml”, which is ˜8 kilobytes and was created on Dec. 6, 2022, is filed herewith by electronic submission and is incorporated by reference herein.

### 1. FIELD

The present disclosure relates generally to the field of diagnostics. The present disclosure provides devices and methods for profiling the RNA fragments bound to ribosomes.

### 2. DESCRIPTION OF RELATED ART

Temporal regulation of gene expression is critical for mammalian embryonic development. Post-transcriptional regulation of maternal transcripts shapes the early gene expression landscape of the mouse embryo due to the absence of transcription from later stages of oocyte maturation through the 2-cell embryo stage (Wang et al., 2001). Consequently, RNA expression and protein abundance are only modestly correlated until the late morula and blastocyst stages, emphasizing the need to elucidate the full scope of post-transcriptional regulation during initial stages of development (Li et al., 2010; Gao et al., 2017; Vastenhouw et al., 2019).

Transcriptome-wide dynamics of ribosome occupancy have remained completely unexplored to date in early mammalian development, despite the development of ribosome profiling techniques over a decade ago (Oh et al., 2000). Ribosome profiling capitalizes on the fact that translating ribosomes protect mRNA fragments with a characteristic sequence length from nuclease digestion (Gebauer et al., 1994). High-throughput sequencing of these ribosome protected fragments (RPFs), or ribosome footprints, provides a snapshot of transcriptome-wide mRNA translation. Importantly, ribosome occupancy correlates better with protein abundance than RNA-seq measurements across diverse systems, including in yeast (Oh et al., 2000) and human cell lines (Ingolia et al., 2009). An even stronger relationship exists between ribosome occupancy and newly synthesized protein abundance (Ingolia et al., 2019; Rogacs et al., 2014), exemplifying the importance of ribosome profiling in investigating translation (Gebauer et al., 1994).

For the last decade, ribosome profiling has been restricted to applications with high input samples, and therefore unavailable for application in the study of early mouse embryonic development. ITP has previously been applied for efficient extraction of nucleic acids from blood, urine, and cell culture samples (Schoch et al., 2009), given its major advantages over conventional RNA extraction approaches such as faster processing times, no requirement of liquid transfers, and high yield with low input RNAs (Khnouf et al., 2019; Han et al., 2019). Despite these advantages, ITP is typically considered to lack the ability to deliver the stringent size selection that would be required for applications such as ribosome profiling (Eid & Santiago, 2017; Abdel-Sayed et al., 2017). A novel method leveraging the principles of microfluidic on-chip isotachophoresis (ITP) for isolation of RPFs would therefore be of great importance.

### SUMMARY

In some aspects, the present disclosure provides methods for detecting nucleic acids such as small nucleic acids including ribosome protected fragments (RPFs). In other aspects, the present disclosure provides devices that may be used in profiling ribosome protected fragments.

In another aspect, the present disclosure provides methods for obtaining one or more nucleic acids comprising:
(A) obtaining a purified cell lysate containing one or more nucleic acid comprising less than 100 pg of the one or more nucleic acids; and
(B) separating the purified cell lysate in a size selection channel to detect one or more nucleic acids.

In some embodiments, the one or more nucleic acids are ribosome protected fragments (RPFs). In some embodiments, the RPFs are RNA. In other embodiments, the one or more nucleic acids are siRNA. In other embodiments, the one or more nucleic acids are microRNA. In other embodiments, the one or more nucleic acids are the degradation products of a cell.

In some embodiments, the one or more nucleic acids comprise from about 10 nucleotides to about 50 nucleotides. In some embodiments, the one or more nucleic acids comprise from about 15 nucleotides to about 40 nucleotides. In some embodiments, the one or more nucleic acids comprise from about 17 to about 35 nucleotides.

In some embodiments, the methods further comprise digesting the obtained purified cell lysate with a nuclease such as an endo exonuclease. In some embodiments, the nuclease is MNase. In some embodiments, the methods further comprise terminating the digestion with a nuclease inhibitor. In some embodiments, the nuclease inhibitor is a ribonucleoside transition metal complex such as a ribonucleoside vanadyl complex. In some embodiments, the methods further comprise terminating the digestion with a chelator such as an aminopolycarboxylic acid. In some embodiments, the chelator is ethylene glycol-bis(β-aminoethyl ether)-N,N,N′,N′-tetraacetic acid.

In some embodiments, the methods further comprise pretreating the size selection channel. In some embodiments, the pretreating comprises pretreating with one or more solutions. In some embodiments, the pretreating comprises treating with a first solution such as a RNA purification solution. In some embodiments, the RNA purification solution is RNaseZAP®. In some embodiments, the pretreating comprises treating with a second solution. In some embodiments, the second solution is water such as nuclease-free water. In some embodiments, the pretreating comprises treating with a third solution. In some embodiments, the third solution is a basic solution. In some embodiments, the basic solution is a hydroxide solution such as an aqueous 1 M NaOH solution. In some embodiments, the pretreating comprises treating with a fourth solution. In some embodiments, the fourth solution is water such as nuclease-free water. In some embodiments, the pretreating comprises treating with a fifth solution. In some embodiments, the fifth solution is an acidic solution. In some embodiments, the acidic solution is a mineral acid solution such as an aqueous 1 M HCl solution.

In some embodiments, the pretreating comprises treating with a sixth solution. In some embodiments, the sixth solution is water such as nuclease-free water. In some embodiments, the pretreating comprises treating with a seventh solution. In some embodiments, the seventh solution is a cross-linking solution. In some embodiments, the cross-linking solution comprises benzophenone. In some embodiments, the cross-linking solution comprises from about 1% w/v to about 20% w/v of the benzophenone such as about 10% w/v benzophenone. In some embodiments, the pretreating comprises treating with a eighth solution. In some embodiments, the eighth solution is an organic solvent such as a C1-C6 alcohol. In some embodiments, the organic solvent is methanol. In some embodiments, the pretreating comprises treating with a ninth solution. In some embodiments, the ninth solution comprises a non-ionic surfactant. In some embodiments, the non-ionic surfactant comprises a hydrophobic component. In some embodiments, the non-ionic surfactant comprises a polyethylene glycol polymer. In some embodiments, the non-ionic surfactant is Triton X-100. In some embodiments, the ninth solution comprises from about 0.01% v/v to about 1% v/v of the non-ionic surfactant. In some embodiments, the ninth solution comprises from about 0.05% v/v to about 0.5% v/v of the non-ionic surfactant. In some embodiments, the ninth solution comprises about 1% v/v of the non-ionic surfactant.

In some embodiments, the methods further comprise loading the size selection channel. In some embodiments, the size selection channel comprises one or more discrete separation zones. In some embodiments, the size selection channel comprises two or more discrete separation zones. In some embodiments, the size selection channel comprises a first separation zone and a second separation zone. In some embodiments, the first separation zone is loaded with a polymer. In some embodiments, the first separation zone consists essentially of a polymer. In some embodiments, the polymer is polyacrylamide. In some embodiments, the first separation zone is loaded with from about 0.1% to about 20% polyacrylamide. In some embodiments, the first separation zone is loaded with from about 5% to about 15% polyacrylamide. In some embodiments, the first separation zone is loaded with about 10% polyacrylamide.

In some embodiments, the size selection channel comprises a second separation zone. In some embodiments, the second separation zone is loaded with a polymer. In some embodiments, the polymer is polyacrylamide. In some embodiments, the polyacrylamide is between about 0.1% and 20% polyacrylamide. In some embodiments, the polyacrylamide is between about 1% and 10% polyacrylamide. In some embodiments, the polyacrylamide is about 5% polyacrylamide.

In some embodiments, the polymer is impregnated with an initiator. In some embodiments, the initiator is photoactivatable. In some embodiments, the initiator is 2,2′-azobis [2-methyl-N-(2-hydroxyethyl) propionamide].

In some embodiments, the methods further comprise initiating the polymerization of the polymer by exposing one or more acrylamide monomers to light. In some embodiments, the light is 200 nm to about 800 nm. In some embodiments, the light is from about 300 nm to about 600 nm such as 365 nm.

In some embodiments, the size selection channel comprises a thickness from about 100 μm to about 500 μm. In some embodiments, the size selection channel comprises a thickness of about 375 μm.

In some embodiments, the methods further comprise adding at least one labeling agent to the purified cell lysate. In some embodiments, at least one of the labeling agents is a nucleic acid. In some embodiments, the labeling agent is DNA, RNA, or dideoxyribonucleic acid. In some embodiments, at least one of the nucleic acids is a dideoxyribonucleic acid. In some embodiments, at least one of the nucleic acids is a ribonucleic acid. In some embodiments, at least one of the labeling agents is unable to be amplified. In some embodiments, at least one of the labeling agents is a 3′-dideoxynucleoside. In some embodiments, at least one of the labeling agents is a 3′-deoxynucleoside.

In some embodiments, at least one of the labeling agents is able to be detected. In some embodiments, the methods comprise monitoring the movement of the labeling agents. In some embodiments, at least one of the labeling agents comprises a fluorescent dye. In some embodiments, the fluorescent dye comprises an emission spectrum from about 300 nm to about 900 nm. In some embodiments, the emission spectrum is from about 400 nm to about 700 nm. In some embodiments, the fluorescent dye is an ATTO dye, an Alexa Fluor dye, a rhodamine dye, or a fluorescein dye. In some embodiments, the fluorescent dye is an ATTO dye.

In some embodiments, the labeling agent migrates through the size selection channel at a rate approximately equivalent to an oligomer from about 5 deoxyribonucleotides to about 35 deoxyribonucleotides. In some embodiments, the oligomer is from about 10 deoxyribonucleotides to about 35 deoxyribonucleotides. In some embodiments, the oligomer is from about 10 deoxyribonucleotides to about 35 deoxyribonucleotides. In some embodiments, the oligomer is from about 15 deoxyribonucleotides to about 25 deoxyribonucleotides. In some embodiments, the oligomer is about 19 deoxyribonucleotides.

In some embodiments, the labeling agent migrates through the size selection channel at a rate corresponding to an oligomer from about 5 deoxyribonucleotides to about 35 deoxyribonucleotides. In some embodiments, the oligomer is from about 20 deoxyribonucleotides to about 75 deoxyribonucleotides. In some embodiments, the oligomer is from about 25 deoxyribonucleotides to about 60 deoxyribonucleotides. In some embodiments, the oligomer is from about 30 deoxyribonucleotides to about 45 deoxyribonucleotides. In some embodiments, the oligomer is about 36 deoxyribonucleotides

In some embodiments, the methods comprise adding two labeling agents to the purified cell lysate. In some embodiments, the two labeling agents comprise a labeling agent that migrates through the size selection channel at a rate approximately equivalent to an oligomer of 19 deoxyribonucleotides. In some embodiments, the two labeling agents comprise a labeling agent that migrates through the size selection channel at a rate approximately equivalent to an oligomer of 36 deoxyribonucleotides. In some embodiments, the two labeling agents are a labeling agents that migrates through the size selection channel at a rate approximately equivalent to an oligomer of 19 and 36 deoxyribonucleotides.

In some embodiments, the purified cell lysate is derived from a sample of about 1 cell to about 1 million cells. In some embodiments, the purified cell lysate is derived from a sample of about 1 cell to about 100,000 cells. In some embodiments, the purified cell lysate is derived from a sample of about 1 cell to about 100 cells. In some embodiments, the purified cell lysate is derived from a sample of about 1 cell.

In some embodiments, the purified cell lysate is of a mammalian cell population. In some embodiments, the mammalian cell population is a human cell population. In some embodiments, the human cell is an embryonic cell population. In other embodiments, the human cell is a FACS sorted human cell population. In other embodiments, the human cell is an immune cell population. In some embodiments, the immune cell population is a population of B cells. In other embodiments, the immune cell population is a population of T cells. In other embodiments, the human cell is a cancer cell population. In some embodiments, the cancer cell population is a population of cancer stem cells.

In some embodiments, the mass of the one or more nucleic acids in the purified cell lysate is less than 80 picograms. In some embodiments, the mass is less than 60 picograms. In some embodiments, the mass is less than 40 picograms. In some embodiments, the mass of the one or more nucleic acids in the purified cell lysate is from about 1 picograms to about 100 picograms. In some embodiments, the mass of the one or more nucleic acids in the purified cell lysate is about 10 picograms to about 80 picograms. In some embodiments, the mass of the one or more nucleic acids in the purified cell lysate is about 40 picograms.

In some embodiments, the separation of the purified cell lysate is by isotachophoresis. In some embodiments, the isotachophoresis comprises applying a current across the size selection channel. In some embodiments, the current is a constant current. In some embodiments, the current is from about 10 mA to about 1 A. In some embodiments, the current is from about 100 mA to about 500 mA such as about 300 mA.

In some embodiments, the isotachophoresis comprises applying a voltage across the size selection channel. In some embodiments, the voltage is from about 0.1 kV to about 10 kV. In some embodiments, the voltage is from about 0.5 kV to about 5 kV such as about 1.1 kV.

In some embodiments, the separation comprises applying the purified cell lysate in a buffer solution. In some embodiments, the buffer solution comprises a buffering agent such as tris or bis-tris. In some embodiments, the buffering agent is bis-tris. In some embodiments, the buffer solution is buffered to a pH of about 6 to about pH of about 8. In some embodiments, the buffer solution is buffered to a pH of about 7 to about pH of about 7.4.

In some embodiments, the buffer solution further comprises a surfactant such as a nonionic surfactant. In some embodiments, the nonionic surfactant comprises an aromatic hydrophobic component. In some embodiments, the aromatic hydrophobic component is 4-2,4,4-trimethylpentylphenyl. In some embodiments, the surfactant further comprises one or more polyethylene glycol or polypropylene glycol repeating units. In some embodiments, the surfactant comprises one or more polyethylene glycol repeating units. In some embodiments, the surfactant comprises from about 5 to about 20 polyethylene glycol repeating units. In some embodiments, the surfactant comprises from about 8 to about 12 polyethylene glycol repeating units. In some embodiments, the surfactant is Triton X-100®. In some embodiments, the buffering solution comprises from about 0.1% w/w to about 10% w/w of the surfactant. In some embodiments, the buffering solution comprises from about 0.25% w/w to about 5% w/w of the surfactant. In some embodiments, the buffering solution comprises from about 0.5% w/w to about 2.5% w/w of the surfactant. In some embodiments, the buffering solution comprises about 1% w/w of the surfactant.

In some embodiments, the buffer solution further comprises a fungicide such as cycloheximide. In some embodiments, buffer solution further comprises a reducing agent. In some embodiments, the reducing agent comprises a pair of thiol groups such as dithiothreitol.

In some embodiments, the buffer solution comprises from about 0.01 mM to about 100 mM of the reducing agent. In some embodiments, the buffer solution comprises from about 0.1 mM to about 10 mM of the reducing agent. In some embodiments, the buffer solution comprises about 1 mM of the reducing agent.

In some embodiments, the buffer solution comprises one or more salts. In some embodiments, the buffer solution comprises a first salt. In some embodiments, the first salt is an alkali earth metal salt. In some embodiments, the first salt is a halide of an alkali earth metal salt such as MgCl₂. In some embodiments, the buffer solution comprises a second salt. In some embodiments, the second salt is an alkali earth metal salt. In some embodiments, the second salt is a halide of an alkali earth metal salt such as CaCl₂). In some embodiments, the buffer solution comprises a third salt. In some embodiments, the third salt is an alkali metal salt. In some embodiments, the third salt is a halide of an alkali metal salt such as NaCl.

In some embodiments, the buffer solution comprises from about 0.1 mM to about 50 mM of the first salt. In some embodiments, the buffer solution comprises from about 0.5 mM to about 25 mM of the first salt. In some embodiments, the buffer solution comprises from about 1 mM to about 10 mM of the first salt. In some embodiments, the buffer solution comprises about 5 mM of the first salt. In some embodiments, the buffer solution comprises from about 0.1 mM to about 50 mM of the second salt. In some embodiments, the buffer solution comprises from about 0.5 mM to about 25 mM of the second salt. In some embodiments, the buffer solution comprises from about 1 mM to about 10 mM of the second salt. In some embodiments, the buffer solution comprises about 5 mM of the second salt. In some embodiments, the buffer solution comprises from about 1 mM to about 2.5 M of the third salt. In some embodiments, the buffer solution comprises from about 25 mM to about 1 M of the third salt. In some embodiments, the buffer solution comprises from about 50 mM to about 500 mM of the third salt. In some embodiments, the buffer solution comprises about 100 mM of the third salt.\

In some embodiments, the separation further comprises adding an electrolyte solution. In some embodiments, the electrolyte solution comprises a polymer such as a polyether polymer. In some embodiments, the polymer is a polyethylene glycol and polypropylene glycol copolymer. In some embodiments, the polymer comprises two polyethylene glycol blocks and a polypropylene glycol block. In some embodiments, the polyethylene glycol block comprises from about 50 to about 250 repeating units. In some embodiments, the polyethylene glycol block comprises from about 75 to about 150 repeating units. In some embodiments, the polyethylene glycol block comprises from about 90 to about 110 repeating units. In some embodiments, the polyethylene glycol block comprises about 101 repeating units. In some embodiments, the polypropylene glycol block comprises from about 10 to about 150 repeating units. In some embodiments, the polypropylene glycol block comprises from about 20 to about 90 repeating units. In some embodiments, the polypropylene glycol block comprises from about 50 to about 60 repeating units. In some embodiments, the polypropylene glycol block comprises about 56 repeating units. In some embodiments, the electrolyte solution comprises from about 2.5% w/w to about 50% w/w of the polymer. In some embodiments, the electrolyte solution comprises from about 10% w/w to about 40% w/w of the polymer. In some embodiments, the electrolyte solution comprises from about 20% w/w to about 30% w/w of the polymer. In some embodiments, the electrolyte solution comprises about 25% w/w of the polymer.

In some embodiments, the electrolyte solution further comprises a buffer. In some embodiments, the buffer is tris or bis-tris. In some embodiments, the bis-tris is bis-tris methane or bis-tris propane. In some embodiments, the electrolyte solution comprises from about 10 mM to about 1 M of the buffer. In some embodiments, the electrolyte solution comprises from about 50 mM to about 500 mM. In some embodiments, the electrolyte solution comprises from about 100 mM to about 300 mM. In some embodiments, the electrolyte solution comprises about 200 mM.

In some embodiments, the methods comprise using a first electrolyte solution and a second electrolyte solution. In some embodiments, the first electrolyte solution further comprises an acid. In some embodiments, the acid is a mineral acid such as hydrochloric acid. In some embodiments, the first electrolyte solution comprises from about 5 mM to about 500 mM of the acid. In some embodiments, the first electrolyte solution comprises from about 25 mM to about 100 mM of the acid. In some embodiments, the first electrolyte solution comprises from about 40 mM to about 60 mM of the acid. In some embodiments, the first electrolyte solution comprises about 50 mM of the acid. In some embodiments, the second electrolyte solution comprise a second buffer. In some embodiments, the second buffer is a buffer that has a buffering point from about pH of 6 to about pH of 8. In some embodiments, the second buffer has a buffering point from about pH of 7 to about pH of 7.4. In some embodiments, the second buffer is MOPS. In some embodiments, the second electrolyte solution comprises from about 10 mM to about 1 M of the second buffer. In some embodiments, the second electrolyte solution comprises from about 50 mM to about 250 mM of the second buffer. In some embodiments, the second electrolyte solution comprises from about 90 mM to about 110 mM of the second buffer. In some embodiments, the second electrolyte solution comprises about 100 mM of the second buffer.

In some embodiments, the separation comprises applying a positive and negative electrode to the size separation channel. In some embodiments, the positive and negative electrodes are applied to separate ends of the size separation channel. In some embodiments, the positive and negative electrodes are applied at opposite ends of the size separation channel. In some embodiments, the first electrolyte solution is applied to the same end of the size separation channel as the positive electrode. In some embodiments, the second electrolyte solution is applied to the same end of the size separation channel as the negative electrode. In some embodiments, the methods comprise stopping the application of current when the longer labeling agent enters the second separation zone. In some embodiments, the methods further comprise emptying a collection well while the current is stopped. In some embodiments, the current is restarted after the well has been emptied. In some embodiments, the current is applied until the shorter labeling agent enters the collection well. In some embodiments, the methods comprise collecting the RPFs in a collection well. In some embodiments, the methods comprise washing the collection well with water. In some embodiments, the water is nuclease-free water. In some embodiments, the collection well is washed twice with water. In some embodiments, the collection well has been filled with a dephosphorylation buffer. In some embodiments, the dephosphorylation buffer comprises a phosphatase. In some embodiments, the dephosphorylation buffer further comprises a buffering agent. In some embodiments, the buffering agent is Tris. In some embodiments, the dephosphorylation buffer further comprises one or more salts. In some embodiments, the salt is NaCl. In some embodiments, the salt is MgCl₂. In some embodiments, the dephosphorylation buffer further comprises NaCl and MgCl₂. In some embodiments, the dephosphorylation buffer further comprises a reducing agent such as dithiothreitol. In some embodiments, the methods further comprise sequencing the one or more nucleic acids. In some embodiments, the methods further comprise quantifying the one or more nucleic acids.

In still yet another aspect, the present disclosure provides methods of obtaining one or more ribosome protected fragments (RPFs) comprising:
(A) obtaining a purified cell lysate containing one or more nucleic acid;
(B) separating the purified cell lysate in a size selection channel to obtain one or more separated ribosome protected fragments.

In some embodiments, the purified cell lysate comprises less than 100 pg of nucleic acid.

In yet another aspect, the present disclosure provides methods of quantifying one or more ribosome protected fragments (RPFs) comprising:
(A) obtaining a purified cell lysate containing one or more nucleic acid;
(B) separating the purified cell lysate in a size selection channel to obtain one or more separated ribosome protected fragments; and
(C) quantifying the separated ribosome protected fragments.

In still yet another aspect, the present disclosure provides methods of determining the sequence of one or more ribosome protected fragments (RPFs) comprising:
(A) obtaining a purified cell lysate containing one or more nucleic acid;
(B) separating the purified cell lysate in a size selection channel to obtain one or more separated ribosome protected fragments; and
(C) sequencing the separated ribosome protected fragments to determine the sequence of the ribosome protected fragments.

In yet another aspect, the present disclosure provides apparatuses for detecting one or more ribosome protected fragments (RPFs), the apparatus comprising:
a reservoir containing a first electrolyte solution;
a reservoir containing a second electrolyte solution; and
a channel, wherein:
   the channel extends between the reservoir containing the first electrolyte solution and the reservoir containing the second electrolyte solution; and
   the channel contains a liquid and a polyacrylamide gel.

In some embodiments, the apparatuses further comprise an elution well. In some embodiments, the first electrolyte solution is a leading electrolyte solution and the second electrolyte solution is a trailing electrolyte solution. In some embodiments, the polyacrylamide gel varies in concentration in the liquid between the reservoir containing the first electrolyte solution and the reservoir containing the second electrolyte solution. In some embodiments, the apparatuses comprise a plurality of reservoirs containing the first electrolyte solution. In some embodiments, the apparatuses comprise:
the plurality of reservoirs containing the first electrolyte solution comprises a first reservoir, a second reservoir and a third reservoir;
the channel contains a cell lysate between the reservoir containing the second electrolyte solution and the first reservoir containing the first electrolyte solution;
the channel contains a first concentration of polyacrylamide gel between the first reservoir containing the first electrolyte solution and the second reservoir containing the first electrolyte solution;
the channel contains a second concentration of polyacrylamide gel between the second reservoir containing the first electrolyte solution and the third reservoir containing the first electrolyte solution; and
the second concentration of polyacrylamide gel is greater than the first concentration of polyacrylamide gel.

In some embodiments, the first concentration of polyacrylamide gel is approximately 5 percent and the second concentration of polyacrylamide gel is approximately 10 percent. In some embodiments, the apparatuses comprise an elution well proximal between the second reservoir containing the first electrolyte solution and the third reservoir containing the first electrolyte solution. In some embodiments, the apparatuses further comprising:
a power supply coupled to a first electrode and a second electrode, wherein:
   the first electrode is located in the reservoir containing the first electrolyte solution; and
   the second electrode is located in the reservoir containing the second electrolyte solution.

In some embodiments, the apparatuses further comprise a control circuit configured to control the power supply. In some embodiments, the control circuit is configured to control the power supply to apply approximately 300 milliamperes (mA) to the channel. In some embodiments, the channel has a thickness of approximately 375 μm. In some embodiments, the channel is formed from polydimethylsiloxane (PDMS).

Other objects, features and advantages of the present disclosure will become apparent from the following detailed description. It should be understood, however, that the detailed description and the specific examples, while indicating preferred embodiments of the invention, are given by way of illustration only, since various changes and modifications within the spirit and scope of the disclosure will become apparent to those skilled in the art from this Detailed Description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B—Schematic of Ribo-ITP. (A) Schematic of the generation of ribosome protected fragments (RPFs). Following RNase digestion, RPFs are isolated with the conventional or novel Ribo-ITP approach. (B) Schematic of the conventional ribosome profiling protocol and the Ribo-ITP process for extraction of RPFs. In Ribo-ITP, marker oligonucleotides with a 5′ fluorophore and 3′ ddC blocking modification, which encapsulate the size range of RPFs, are added to the digested cellular lysate. Lysate contents are loaded into the channel (t₀), then an electrical current is applied to selectively focus species of a specific electrophoretic mobility range, enabling nucleic acid extraction by isotachophoresis. Nucleic acids are extracted in a narrow ITP band, and then size selected as they migrate through 5% (t₁) and 10% (t₂) polyacrylamide gels, respectively. At the end of the run, purified and size-selected RNAs are collected (t₃).

FIGS. 2A-B—Channel design and dimensions. (A) The top view of the ITP chip layout designed with SOLIDWORKS (units in mm). The design was 3-D printed to be used as a mold for microchannels. The thickness of the channel features was 375 μm and that of the rectangular base was 1.5 mm. Linear tapering was applied from the rectangular base to the outer edge (rounded rectangle). (B) Microfluidic device setup; indicating lysate, extraction, and size-selection channels; trailing electrolyte (TE) and leading electrolyte (LE) reservoirs (1-3); and elution well. Buffers corresponding to each channel and reservoir are color coded. Marker oligonucleotide fluorescence is denoted by green.

FIG. 3—Verification of encapsulation of typical RPF size range by fluorescent DNA marker oligonucleotides. The gel image displays the relative mobilities of small RNAs and modified fluorescent markers used in Ribo-ITP. The Zymo Research R1090 small RNA ladder (Z) and synthetic RNA oligonucleotides(S) exemplify potential fragment lengths generated by MNase digestion. 19 nt and 36 nt fluorescent DNA oligonucleotide markers (M) are used in Ribo-ITP experiments.

FIG. 4—Conductivity and pH measurements of dephosphorylation buffer subjected to Ribo-ITP. The first step of library preparation in Ribo-ITP is 3′ dephosphorylation. Given that pH is an important factor determining dephosphorylation efficiency45, we determined the impact of Ribo-ITP collection on the conductivity and pH of the dephosphorylation buffer. We found negligible pH change (right axis, blue) and only a 11.0%+1.83% (SEM) change in conductivity (left axis, green) for the collection distance (5 mm, denoted by the vertical line) used in Ribo-ITP.

FIGS. 5A-B—Yield comparison between Ribo-ITP and conventional gel extraction. (A) Representative gel images of control inputs (I), Ribo-ITP elutions (R), and gel extraction (G) samples. Four RNA species (17, 21, 25, and 29 nt) were used with total inputs of 20 and 40 ng. Fluorescent marker oligonucleotides were spiked into control and gel extraction samples prior to gel visualization. (B) Gel image quantification of control inputs (gray), Ribo-ITP elutions (orange), and gel extraction (purple) samples. Minimum, maximum, and average values are represented by the box and the horizontal bar. Only the 25 and 29 nt RNA marker bands were quantified for the yield calculation.

FIGS. 6A-D—Characterization of Ribo-ITP method and validation of efficacy in ultra-low input ribosome profiling. (A) Representative gel images highlighting inputs (I), RNAs recovered by Ribo-ITP (R), and gel electrophoresis (G) are shown. Four RNAs of 17, 21, 25, and 29 nt used in the experiment were radioactively labeled at their 5′end. Percent yield was calculated for the 25 nt RNA. (B) Representative gel image of a size selection experiment. 100 ng of MNase-digested RNA from K562 cells (D) was used as an input for Ribo-ITP after the addition of the two fluorescent marker oligonucleotides (I). In a typical experiment, we collect the sample flanked by the two fluorescent nucleotide markers (Fraction 2 here). Here, we also collected the RNAs that eluted before the arrival of the shorter fluorescent marker (Fraction 1) as well as the RNAs that were located behind the longer fluorescent marker (Fraction 3), which typically remain in the channel. The percent yield of RNAs larger than the longer fluorescent marker oligonucleotide (>˜36 nt) (blue) and RNAs flanked by the markers (orange), corresponding to the size range of RPFs, are plotted for each fraction. (C) Schematic of the sequencing library preparation protocol. In a single-tube reaction, isolated RPFs are 3′ dephosphorylated and poly(A)-tailed. A template-switching reverse transcriptase (RT) creates templates that incorporate UMI-containing adapters. (D) Pairwise correlation of gene-level ribosome occupancy measured in conventional ribosome profiling and Ribo-ITP from human K562 cells. The left plot highlights two replicates of conventional ribosome profiling experiments from ˜10M cells. The middle plot is from two replicates of Ribo-ITP with ˜100 cells. For the plot on the right, we used the mean number of counts per million reads for each gene. The Spearman correlation coefficients between the gene-level ribosome occupancies are indicated on the top left corner.

FIGS. 7A-B—Ribo-ITP enables efficient RNA extraction from cell lysates. (A) Inputs (I, gray) were prepared by adding 40 ng of RNA to lysates from ˜1,000 K562 cells. The RNA consisted of four species ranging from 17 to 29 nt in length. Fluorescent marker DNAs were added to Ribo-ITP samples (R) in addition to EGTA (10 mM). RNA extraction and isolation was done with Ribo-ITP followed by visualization using gel electrophoresis. (B) Yield of the 25 and 29 nt RNAs was quantified and plotted for two replicates (84% and 91%, 20 respectively).

FIG. 8—Mean to dispersion relationship for Ribo-ITP and conventional ribosome profiling Transcripts with at least one count per million (cpm) in at least two out of three replicates were selected. Log₂ of mean cpm (x-axis) was plotted against the square root of the standard deviation of cpm values (y-axis). Green (Ribo-ITP from ˜100 cells) and red (conventional ribosome profiling from ˜10M cells) points represent individual transcripts with mean log₂(cpm) greater than two.

FIGS. 9A-B—Metagene plots and footprint length distribution for Ribo-ITP from ˜100 cells compared to conventional ribosome profiling from ˜10M cells. (A) Metagene plots of Ribo-ITP versus conventional ribosome profiling in human K562 cells were shown. Position 0 corresponds to the start (left, light green) or stop (right, dark green) site. Aligned positions of ribosome footprints were adjusted according to their A-site offsets. One representative replicate for each method is plotted. (B) The mean percentage of specific read lengths among the total mapped reads is plotted. Ribbons around the lines represent standard error of the mean.

FIGS. 10A-B—Region Counts for Ribo-ITP from ˜100 cells compared to conventional ribosome profiling from ˜10M cells. (A) Percentages of ribosome profiling reads aligning to different transcript regions are plotted. The percentage of reads mapped to the CDS is indicated for each experiment. (B) For each transcript, we multiplied its region length by the total number of ribosome footprints for the given experiment. The plotted overall percentage corresponds to the sum of these weighted counts across transcripts.

FIGS. 11A-E—Ribo-ITP enables single cell and single embryo measurements of ribosome occupancy. (A) Schematic of the mouse experiments. Unfertilized oocytes (GV- and MII-stage) from C57BL/6J strain along with zygotes to the 8-cell stage embryos from a crossbreed of two strains (C57BL/6J and CAST/EiJ) were collected for RNA expression and ribosome occupancy measurements. (B) Ribosome occupancy around the translation start and stop sites in a representative zygote (1-cell) and an 8-cell stage embryo. Translation start (or stop) sites are denoted by the position 0. Aggregated read counts (y-axis) relative to the start (or stop) sites are plotted after A-site correction. (C) On the left, the distribution of reads across transcript regions (5′ UTR, CDS and 3′UTR) are shown. On the right, the distribution of the lengths of these regions weighted by their ribosome occupancy are depicted. The error bars indicate the standard error of the mean percentages. (D) Pairwise correlation of gene-level ribosome occupancy in single cells are plotted along with Spearman correlation coefficients (top left). (E-F) The standard error and mean of centered log ratio of the ribosome occupancy (y-axis) was plotted for representative transcripts that were previously shown to have increased polysome association in GV- (panel E) or MII-stage (panel F) oocytes (Deng et al., 2014) (remaining genes are shown in FIG. 15).

FIG. 12—Number of genes detected as a function of CDS mapping UMIs. The number of reads (x-axis) is plotted against the number of detected genes (y-axis). Three representative replicates are shown for each stage of development used for single cell ribosome profiling experiments. The total number of detected genes using all CDS mapping reads is indicated along with genes detected with 5k, 10k, 20k, 30k and 40k sub-sampled coding regions mapping UMIs.

FIGS. 13A-B—Metagene plots for mouse Ribo-ITP and RNA-Seq experiments. (A) Metagene plots of translation start and stop sites from a representative Ribo-ITP experiment using a 2-cell or a 4-cell stage mouse embryo. Start sites (light green, left) and stop sites (dark green, right) are at position 0 on the x-axis. Positions of the aligned reads are adjusted according to their A-site offsets. (B) Metagene plots of translation start and stop sites from RNA-Seq data. GEO accession numbers of the experiments are indicated on the plots. In contrast to the ribosome profiling data, there is no detectable peak is observed at translation start or stop sites.

FIG. 14—Pairwise correlation of read counts from Ribo-ITP and RNA-Seq experiments. CDS-mapping read counts from each transcript were used to compute the Spearman correlation coefficient. Ribo-ITP (orange) and RNA-Seq (blue) experiments are ordered by developmental stage. The colors indicate the strength of the correlation.

FIGS. 15A-B—Ribosome occupancy in GV- and MII-stages of transcripts with previously identified differential polysome association. (A) The mean of centered log-ratio of ribosome occupancy (y-axis) was plotted along with the standard error of the mean. These transcripts were identified as having increased polysome association in the MII-stage compared to GV-stage (Deng et al., 2014) (B) Similar to panel (A) with the exception that these transcripts were found to display decreased polysome association in the MII-stage compared to the GV-stage (Deng et al., 2014).

FIGS. 16A-E—Allele specific translation and RNA expression in early mouse development. (A) Strain-specific SNPs were used to assign sequencing reads to the paternal and maternal allele (Example 6). Standard error and mean of the percentage of paternal reads in each stage (y-axis) is plotted. (B,D,E) Line-plots (top) indicate the percentage of paternal reads (y-axis) in RNA-Seq and Ribo-ITP experiments. The reads are combined across replicates and error bars indicate standard error of the mean of paternal ratios. At the bottom, normalized maternal and paternal reads counts are plotted for all individual replicates and SNPs. The total number of detected coding SNPs and their corresponding colors are shown with color scales. (C) The percentage of paternal reads is indicated by different shades of gray for ribosome occupancy (upper triangles with orange borders) and RNA expression (lower triangles with blue borders). Genes that displayed differential ribosome engagement in an allele-specific manner in comparison to RNA expression were grouped into four clusters. The prototypical shared pattern in each cluster is displayed on the right.

FIGS. 17A-B—Distribution of sequencing reads that overlap strain-specific SNPs. (A) Ribosome footprints that overlap strain-specific SNPs were used to determine the percentage of reads that match the maternal (green) and paternal (red) allele. A small percentage of reads differed from either allele and are labeled as “other” (dark blue). (B) Reads from RNA-seq experiments were used as in panel (A).

FIGS. 18A-B—Allele-specific ribosome occupancy and RNA expression of genes with the highest number of informative reads (A) The percentage of ribosome footprints that originate from the paternal allele was visualized for genes with at least 10 allele-specific reads at each stage. (B) The percentage of RNA sequencing reads that were assigned to the paternal allele was plotted for the set of genes in panel (A).

FIGS. 19A-B—Representative genes with no allele-specific differences between RNA expression and ribosome occupancy. Line-plots (top) indicate the percentage of paternal reads (y-axis) in RNA-Seq and Ribo-ITP experiments. The reads are combined across replicates and error bars indicate standard error of the mean of paternal ratios. At the bottom, normalized maternal and paternal reads counts are plotted for all individual replicates and SNPs. The total number of detected coding SNPs and their corresponding colors are shown with color scales. Each vertical bar corresponds to a replicate experiment.

FIGS. 20A-F—Representative genes with allele-specific difference in ribosome occupancy and RNA expression. Similar to FIG. 19. (A) Representative gene from cluster II with allele-specific ribosome occupancy bias (e.g. of maternal bias). (B,C) Representative genes from cluster III with delayed engagement of ribosomes in allele- and stage-specific manner i.e. parental RNA expression is observed at or before the 4-cell stage, yet these RNAs predominantly engage with ribosomes only in the 8-cell stage. (D) Representative genes from cluster IV with differential ribosome occupancy compared to RNA expression in 8-cell stage and (E,F) in 4-cell stage.

FIGS. 21A-E—Differential translation efficiency between developmental stages and association between ribosome occupancy and protein abundance. (A) 50 genes with the highest variability in ribosome occupancy across developmental stages is plotted (standardized variance >4.8; Example 6). The colors represent the average of the centered log ratio of the mean of ribosome occupancy where average is taken across replicates. (B) Volcano plots depict the statistical significance (y-axis) and log₂ fold-change (x-axis) in translation efficiency between two developmental stages (Example 6). Colored points indicate transcripts with significant differences (FDR<0.01). (C) The centered log-ratio normalized read counts from Ribo-ITP and RNA-seq experiments are plotted for the highlighted genes. All replicate measurements from the given developmental stage are shown. (D,E) Sankey diagrams depict the relationships between protein abundance with RNA expression and ribosome occupancy. The color and thickness of the links connecting the nodes are proportional to the strength of the Spearman rank correlation (Example 6).

### DESCRIPTION OF ILLUSTRATIVE EMBODIMENTS

The present disclosure provides methods of analyzing the nucleic acids bound to the ribosomes. The present disclosure provides ways to obtain and determine the expression of these nucleic acids in one or more cells such as embryonic cells, stem cells, cancer cells, or immune cells. These methods relate to the using of the electrophoresis on a chip to separate the nucleic acids bound to the ribosome from other cellular nucleic acids. The present disclosure also provides devices that may be used to analyze the ribosomes.

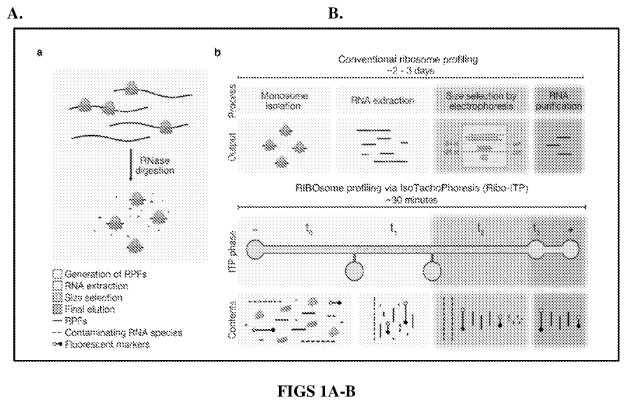

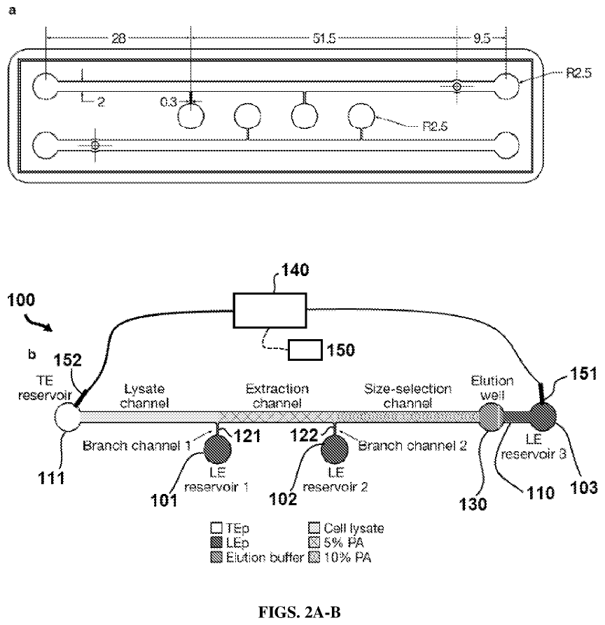

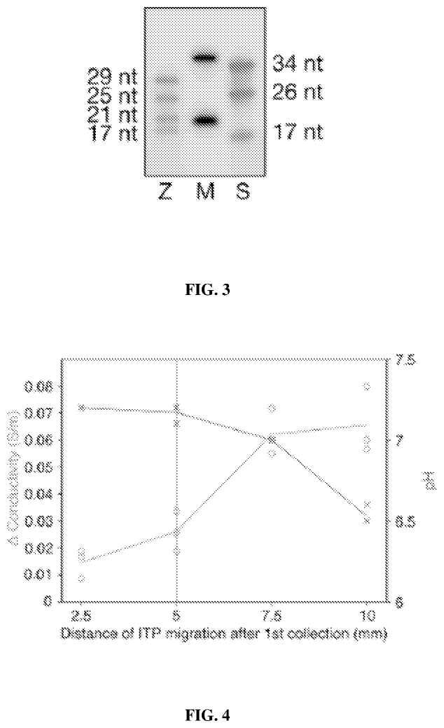

Here, the present disclosure provides microfluidic polydimethylsiloxane (PDMS) chips designed and manufactured to recover ribosome footprints from nuclease-digested lysates with high yield using a specialized technique named RIBOsome profiling via IsoTachoPhoresis (Ribo-ITP) (FIGS. 1A-B, FIGS. 2A-B). This system implements several modifications to the traditional chemistry to achieve single-cell ribosome profiling by coupling ITP with an on-chip size selection. In particular, pretreatment of the channel with benzophenone enabled light-induced polymerization of polyacrylamide inside PDMS chips (McGlincy & Ingolia, 2017). To aid visualization, DNA oligonucleotide markers containing a 5′ fluorophore and 3′ dideoxycytosine modification were included to prevent marker amplification in downstream library preparation (FIG. 3). An on-chip buffer exchange allowed the purified RNAs to be directly compatible with 3′ dephosphorylation, the first step in sequencing library preparation (FIG. 4). Finally, an efficient single tube library preparation chemistry that relies on a template switching reverse transcriptase and incorporation of unique molecular indexes (UMIs) at the 5′ end of the RPFs was adopted. Collectively, these optimizations reduced sample requirements by many orders of magnitude in order to deliver ribosome occupancy measurements from ultra-low input samples, including single cells. In the conventional ribosome profiling method, RPFs are isolated by phenol-chloroform based RNA extraction followed by size selection using polyacrylamide gel electrophoresis (Green & Sambrook, 2019). Given that a typical mammalian cell contains ˜10-40 pg of RNA, an approach capable of generating ribosome occupancy measurements from such limiting amounts needs to maintain consistently high yield of RPF recovery with inputs in the picogram range.

### I. Isotachophoresis and Methods of Separating Nucleic Acids

In some embodiments, the present disclosure provides methods of using electrophoresis such as isotachophoresis to separate nucleic acids of different sizes. In some embodiments, the methods use isotachophoresis. Isotachophoresis is a form of electrophoresis that utilizes a discontinuous buffer system. In particular, the present methods may contemplate using one or more distinct electrolyte solutions. In particular, the methods may utilize a first or leading electrolyte solution and a second or trailing electrolyte solution. The first electrolyte solution contains one or more ions that have a high ionic mobility while the second electrolyte solution contains one or more ions that have a low ionic mobility.

The isotachophoresis may be carried out in a size selection channel that comprises a polymer such as polyacrylamide. The size selection channel comprises one or more separation zones. In particular, the channel may comprise one, two, three, four, or more separation zones. Within the separation zones, the amount of the polymer differs. In particular, the amount of the polymer in the first separation zone may be loaded from about 0.1% to about 20%, from about 5% to about 15%, or from about 8% to about 12%. In some embodiments, the amount of the polymer in the first separation zone is from about 0.1%, 0.5%, 1%, 2%, 3%, 4%, 5%, 6%, 7%, 8%, 9%, 10%, 11%, 12%, 13%, 14%, 15%, 16%, 17%, 18%, 19%, to about 20%, or any range derivable therein. The amount of the polymer in the first separation zone may be about 10%. In some embodiments, the size selection channel may comprise a second separation zone. In particular, the amount of the polymer in the second separation zone may be loaded from about 0.1% to about 20%, from about 1% to about 10%, or from about 4% to about 6%. In some embodiments, the amount of the polymer in the second separation zone is from about 0.1%, 0.5%, 1%, 2%, 3%, 4%, 5%, 6%, 7%, 8%, 9%, 10%, 11%, 12%, 13%, 14%, 15%, 16%, 17%, 18%, 19%, to about 20%, or any range derivable therein. The amount of the polymer in the second separation zone may be about 5%. These solutions may further comprise a denaturing agent such as urea. The amount of denaturing agent in the solution may be from about 1 M to about 20 M, from about 4 M to about 10 M, from about 6 M to about 9 M. The amount of the denaturing agent may be from about 500 mM, 1 M, 2 M, 3 M, 4 M, 5 M, 6 M, 7 M, 8 M, 9 M, 10 M, 11 M, 12 M, 13 M, 14 M, 15 M, 16 M, 17 M, 18 M, 19 M, to about 20 M, or any range derivable therein.

Each of these separation zones may be further treated with one or more solutions. These separation zones may be treated with water, such as deionized and/or nuclease-free water, an acid solution, a basic solution, or a cross-linking agent. In some embodiments, the cross-linking agent may be benzophenone or similar cross-linking agent. The cross-linking agent may be from about 1% w/v to about 25% w/v, from about 5% w/v to about 20% w/v, or from about 10% w/v to about 15% w/v. The amount of benzophenone may be from about 1% w/v, 2% w/v, 3% w/v, 4% w/v, 5% w/v, 6% w/v, 7% w/v, 8% w/v, 9% w/v, 10% w/v, 11% w/v, 12% w/v, 13% w/v, 14% w/v, 15% w/v, 16% w/v, 17% w/v, 18% w/v, 19% w/v, 20% w/v, to about 25% w/v, or any range derivable therein. The benzophenone solution may be in an organic solvent. The organic solvent may be a polar aprotic solvent such as acetone, acetonitrile, or dimethyl sulfoxide.

In some aspects, the leading or first electrolyte solution comprises a polymer. The leading electrolyte solution may further comprise a buffer in water. The water used in the electrolyte solutions may be purified, in particular, the water should be free from any nuclease. In particular, the polymer may further comprise one or more polypropylene glycol and one or more polyethylene glycol units. The polyethylene glycol unit of the polymer may comprise from about 50 to about 250 repeating units, from about 75 to about 150 repeating units, or from about 90 to about 110 repeating units. In some embodiments, the polyethylene glycol unit of the polymer comprises from about 50, 60, 70, 80, 85, 90, 95, 96, 98, 100, 102, 104, 105, 110, 120, 130, 140, to about 150 repeating units; or any range derivable therein. Similar, the polypropylene glycol unit of the polymer may comprise from about 10 to about 150 repeating units, from about 20 repeating units, or from about 50 to about 60 repeating units. In some embodiments, the polyethylene glycol unit of the polymer comprises from about 10, 20, 30, 40, 50, 52, 54, 55, 56, 58, 60, 70, 80, 90, 100, 110, 120, 130, 140, to about 150 repeating units, or any range derivable therein. The polymer may further comprise at two or more polyethylene glycol repeating units. In particular, the polymer may comprise two polyethylene glycol unit and a polypropylene glycol unit. The polymer may comprise from about 2.5% w/w to about 50% w/w, from about 10% w/w to about 40% w/w, or from about 20% w/w to about 30% w/w of the solution. In some embodiments, the solution comprises from about 5%, 10%, 15%, 16%, 18%, 20%, 22%, 24%, 25%, 26%, 28%, 30%, 35%, 40% 45%, to about 50% w/w, or any range derivable therein.

In some embodiments, the electrolyte solutions comprise a buffer. The buffer may be a buffer that maintains the solution at a pH from about 6 to about 8, from about 6.5 to about 7.5, or from about 7 to about 7.4. The pH may be from about 6, 6.2, 6.4, 6.6, 6.8, 7, 7.2, 7.4, 7.6, 7.8, to about 8, or any range derivable therein. The buffer may be tris, bis-tris, or a similar buffer that buffers at those pH's. In particular, the electrolyte solutions may further comprise one or more buffers at a concentration from about 10 mM to about 1 M, from about 50 mM to about 500 mM, or from about 100 mM to about 300 mM. The concentration of the buffer in the electrolyte solution is from about 10 mM, 30 mM, 50 mM, 70 mM, 80 mM, 100 mM, 120 mM, 140 mM, 160 mM, 180 mM, 200 mM, 220 mM, 240 mM, 260 mM, 280 mM, 300 mM, 320 mM, 340 mM, 360 mM, 380 mM, 400 mM, 425 mM, 450 mM, 475 mM, 500 mM, 600 mM, 700 mM, 800 mM, 900 mM, or 1 M, or any range derivable therein.

In some embodiments, the electrolyte solution may further comprise a second buffer. The second buffer may be a buffer that maintains the solution at a pH from about 6 to about 8, from about 6.5 to about 7.5, or from about 7 to about 7.4. The pH may be from about 6, 6.2, 6.4, 6.6, 6.8, 7, 7.2, 7.4, 7.6, 7.8, to about 8, or any range derivable therein. The second buffer may be HEPES, MOPS, or a similar buffer that buffers at those pH's. In particular, the electrolyte solutions may further comprise one or more second buffers at a concentration from about 10 mM to about 1 M, from about 50 mM to about 500 mM, or from about 100 mM to about 300 mM. The concentration of the second buffer in the electrolyte solution is from about 10 mM, 30 mM, 50 mM, 60 mM, 70 mM, 80 mM, 90 mM, 100 mM, 110 mM, 120 mM, 130 mM, 140 mM, 160 mM, 180 mM, 200 mM, 220 mM, 240 mM, 260 mM, 280 mM, 300 mM, 320 mM, 340 mM, 360 mM, 380 mM, 400 mM, 425 mM, 450 mM, 475 mM, 500 mM, 600 mM, 700 mM, 800 mM, 900 mM, or 1 M, or any range derivable therein.

In some embodiments, the electrolyte solution may further comprise an acid. The acid may be a mineral acid such as hydrochloric acid, hydrobromic acid, or hydroiodic acid. In some embodiments, the acid has a pKₐ of less than 7, less than 5, less than 3, less than 1, less than 0, or less than −5. In some embodiments, the concentration of acid in the electrolyte solution may be from about 5 mM to about 500 mM of the acid, from about 25 mM to about 100 mM of the acid, or from about 40 mM to about 60 mM. The concentration of the acid is from about 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 150 mM, 200 mM, 250 mM, 300 mM, 350 mM, 400 mM, 450 mM, or 500 mM, or any range derivable therein.

The methods may also further comprise a buffer solution. The buffer solution comprises one or more buffering agent such as Tris or bis-tris. The buffering agent may be a buffer that maintains the solution at a pH from about 6 to about 8, from about 6.5 to about 7.5, or from about 7 to about 7.4. The pH may be from about 6, 6.2, 6.4, 6.6, 6.8, 7, 7.2, 7.4, 7.6, 7.8, to about 8, or any range derivable therein. In particular, the buffer solution may further comprise one or more buffering agent at a concentration from about 10 mM to about 1 M, from about 50 mM to about 500 mM, or from about 100 mM to about 300 mM. The concentration of the second buffer in the electrolyte solution is from about 10 mM, 30 mM, 50 mM, 60 mM, 70 mM, 80 mM, 90 mM, 100 mM, 110 mM, 120 mM, 130 mM, 140 mM, 160 mM, 180 mM, 200 mM, 220 mM, 240 mM, 260 mM, 280 mM, 300 mM, 320 mM, 340 mM, 360 mM, 380 mM, 400 mM, 425 mM, 450 mM, 475 mM, 500 mM, 600 mM, 700 mM, 800 mM, 900 mM, or 1 M, or any range derivable therein.

The buffer solution may further comprise one or more components. One of those components may be a surfactant such as a nonionic surfactant. The nonionic surfactant may further comprise a hydrophobic component and a polyethylene glycol component. The hydrophobic component may further comprise one or more aromatic hydrophobic components such as 4-2,4,4-trimethylpentylphenyl. The polyethylene glycol component may further comprise one or more polyethylene glycol repeating units. The polyethylene glycol component may comprise from about 5 to about 20 repeating units or from about 8 to about 12 repeating units. The buffer solution may comprise from about 0.1% w/w to about 10% w/w, from about 0.25% w/w to about 5% w/w, or from about 0.5% w/w to about 2.5% w/w. The buffer solution may comprise from about 0.1% w/w, 0.25% w/w, 0.5% w/w, 1% w/w, 2% w/w, 2.5% w/w, 3% w/w, 4% w/w, 5% w/w, 6% w/w, 7% w/w, 8% w/w, 9% w/w, to about 10% w/w, or any range derivable therein.

The buffer solution may further comprise one or more therapeutic agents such as a bactericide or bacteriostatic agent or a fungicide. Furthermore, the buffer solution may further comprise one or more reducing agents. The reducing agent may be a sulfur containing compound such as dithiothreitol or a vitamin such as vitamin E or C. The amount of reducing agent in the buffer solution may be from about 0.01 mM to about 100 mM, from about 0.1 mM to about 10 mM, or form about 0.5 mM to about 5 mM. In some embodiments, the amount of reducing agent in the buffer solution may be from about 0.01 mM, 0.05 mM, 0.1 mM, 0.25 mM, 0.5 mM, 0.75 mM, 1 mM, 5 mM, 10 mM, 20 mM, 40 mM, 50 mM, 60 mM, 80 mM, to about 100 mM, or any range derivable therein.

Finally, the buffer solution may further comprise one or more salts. For example, the salt may be an alkali salt or an alkali earth salt such as a sodium, lithium, potassium, magnesium, or calcium. These salts may be halide such as a chloride, bromide, or iodide. The buffer solution may further comprise sodium chloride, magnesium chloride, or calcium chloride. The buffer solution may further comprise sodium chloride, magnesium chloride, and calcium chloride. The amount of each salt in the buffer solution may be from about 0.01 mM to about 100 mM, from about 0.1 mM to about 10 mM, or form about 0.5 mM to about 5 mM. In some embodiments, the amount of each salt in the buffer solution may be from about 0.01 mM, 0.05 mM, 0.1 mM, 0.25 mM, 0.5 mM, 0.75 mM, 1 mM, 5 mM, 10 mM, 20 mM, 40 mM, 50 mM, 60 mM, 80 mM, to about 100 mM, or any range derivable therein.

In some embodiments, the methods may further comprise one or more other solutions. These solutions may further comprise one or more buffers, acids, salts, or polymers. These solutions may further comprise one or more polymer such as a poloxamer or another non-ionic polymer such as polyvinylpyrrolidone. These solutions may further comprise one or more polymers. The amount of the polymer in the solution may be from about 0.01% to about 5%, from about 0.05% to about 2.5%, or from about 0.05% to about 1%. The amount of polymer in the solution may be from about 0.01%, 0.025%, 0.05%, 0.075%, 0.1%, 0.25%, 0.5%, 0.75%, 1%, 2%, 3%, 4%, to about 5%, or any range derivable therein.

While these methods may be used with any amount of nucleic acids from a cell lysate, the methods are particularly useful for small amounts of nucleic acids. In particular, the methods may be used with less than 1 ng of the nucleic acids, less than 500 pg of the nucleic acids, less than 250 pg of the nucleic acids, less than 100 pg of the nucleic acids, less than 75 pg of the nucleic acids, less than 50 pg of the nucleic acids, less than 25 pg of the nucleic acids, or less than 10 pg of the nucleic acids. The amount of nucleic acids may be from about 1 pg to about 1 ng, from about 5 pg to about 500 pg, from about 10 pg to about 100 pg, or from about 10 pg to about 50 pg of the nucleic acids. The amount of nucleic acids is from about 1 pg, 2 pg, 4 pg, 6 pg, 8 pg, 10 pg, 15 pg, 20 pg, 25 pg, 30 pg, 35 pg, 40 pg, 45 pg, 50 pg, 60 pg, 70 pg, 80 pg, 90 pg, 100 pg, 150 pg, 200 pg, 250 pg, 300 pg, 400 pg, 500 pg, 600 pg, 700 pg, 750 pg, 800 pg, 900 pg, to about 1 ng, or any range derivable therein. The amount of nucleic acid might be the amount that is contained within at least 1,000 cells, 500 cells, 100 cells, 50 cells, 10 cells, 5 cells, or 1 cell. The amount of nucleic acids is from about 1 cell to about 1000 cells, from about 1 cells to about 100 cells, or from about 1 cells to about 10 cells.

### II. Ribosomes, Nucleic Acids, and Ribosome Protected Fragments

The present disclosure relates to methods of analyzing the ribosomes of a cell or a population of cells. The cell population may be any mammalian cell such as human cells. The human cell may be an embryonic cell, a cancer cell, a stem cell, or an immune cell. The ribosomes in these cells are used to prepare and convert mRNA into a growing amino acid chain or a polypeptide. In some embodiments, the present disclosure relates to methods of analyzing the nucleic acid fragments protected by the ribosomes from nuclease digestion. In some embodiments, the ribosome protected fragments may be generated after treatment of cells with certain chemicals such as small molecules. In some embodiments, the cells may be pretreated with translation inhibitors such as cycloheximide, lactimidomyci, or harringtonine. The nucleic acids fragments protected by ribosomes may be used to determine the expression of certain proteins and gene products. Furthermore, the present disclosure provides methods of analyzing one or more nucleic acids.

These nucleic acids may include degradation products including degradation products from a cellular process. The nucleic acids may be short nucleic fragments like micro RNAs, small RNAs, or piRNAs. These nucleic acids may comprise less than 250 nucleotides in length, less than 200 nucleotides, less than 150 nucleotides, less than 100 nucleotides, less than 75 nucleotides, less than 50 nucleotides, less than 40 nucleotides less than 35 nucleotides, less than 30 nucleotides, less than 25 nucleotides or less than 20 nucleotides. In some embodiments, the nucleic acids may have a length from about 5 nucleotides to about 50 nucleotides, from about 10 nucleotides to about 40 nucleotides, from about 15 nucleotides to about 35 nucleotides, or from about 15 nucleotides to about 30 nucleotides. The nucleic acids measured using these methods may have a length from about 5, 10, 15, 20, 25, 30, 35, 40, 45, 50, 60, 70, 75, 80, 90, 100, 125, 150, 175, 200, 225 to about 250 nucleotides, or any range derivable therein.

### III. Definitions

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

As used herein, the term “patient” or “subject” refers to a living mammalian organism, such as a human, monkey, horse, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo [2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Conventional Ribosome Profiling Comparison

In the conventional ribosome profiling method, RPFs are isolated by phenol-chloroform based RNA extraction followed by size selection using polyacrylamide gel electrophoresis (Green & Sambrook, 2019). Given that a typical mammalian cell contains ˜10-40 pg of RNA, an approach capable of generating ribosome occupancy measurements from such limiting amounts needs to maintain consistently high yield of RPF recovery with inputs in the picogram range.

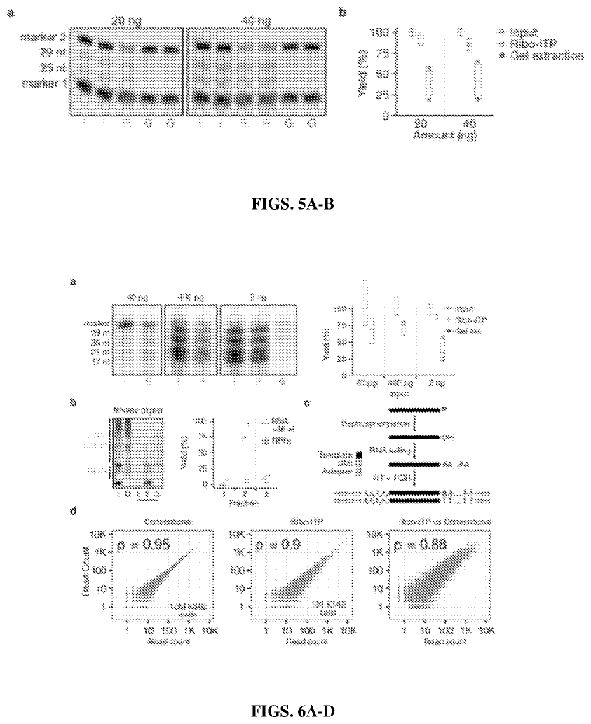

The recovery of RNAs that span the typical size range of RPFs (˜21-35 nt) achieved by the conventional method (Gerashchenko & Gladyshev, 2016) was first compared to the approach of the inventors, Ribo-ITP. For 20 ng input samples, Ribo-ITP yielded 94±3.5% (SEM) recovery in contrast to only 38±10.9% (SEM) for the conventional gel extraction approach (FIGS. 5A-B). The recoveries from ultra-low RNA inputs (40 pg-2 ng) were visualized and quantified with a radioactive labeling assay. With a 2 ng RNA input, 87.5±3.2% yield was achieved by Ribo-ITP compared to 35.3±11.4% by conventional gel extraction (FIG. 2A). When RNA inputs were decreased further to 400 pg and 40 pg, the recovery by Ribo-ITP remained high at 74±6.1% and 67.5±10.6%, respectively (FIG. 6A). Gel extraction was observed to have negligible yield with these samples. Thus, the consistently high RNA yields obtained with Ribo-ITP demonstrated that this method empowers high yield extraction even at ultra-low inputs.

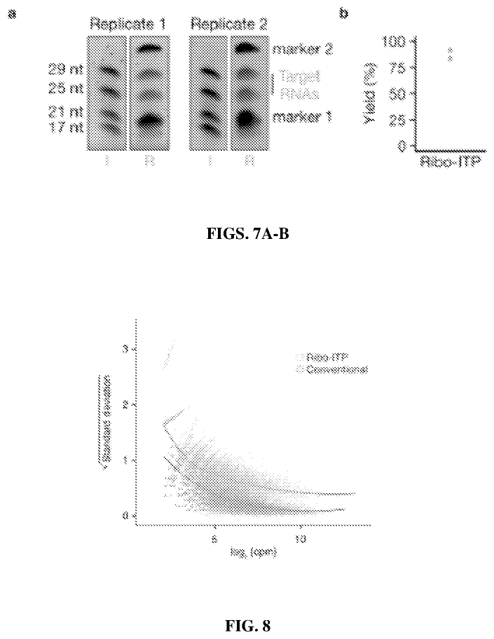

To analyze the efficiency of the method of the inventors at excluding RNA fragments larger than RPFs (>36 nt), total RNA from a human myelogenous leukemia cell line (K562) was digested with micrococcal nuclease (MNase). The sample was purified and subjected to Ribo-ITP (FIG. 6B). Exclusion of 94% of the unwanted large RNA fragments (>36 nt) (FIG. 6B) was achieved. Finally, to verify the ability of Ribo-ITP to extract RNAs from complex cellular lysates, RPF-sized synthetic RNAs (17, 21, 25, 29 nt) were spiked into total cellular lysates from ˜1000 K562 cells. Ribo-ITP of this sample recovered the spiked RNAs with stringent size selection and high yield (FIG. 7). Collectively, these results indicated that Ribo-ITP was able to simultaneously extract and size-select RPF-size RNAs from cellular lysates with high yield.

Referring back now to FIG. 2A, an apparatus 100 for detecting one or more ribosome protected fragments (RPFs) comprises a first reservoir 101, a second reservoir 102, a third reservoir 103, a fourth reservoir 111 and a channel 110 extending between the reservoirs. In specific embodiments first reservoir 101 is in fluid communication with channel 110 via a channel 121, and second reservoir 102 is in fluid communication with channel 110 via a channel 122.

The embodiment shown also comprises an elution well 130 between second reservoir 102 and third reservoir 103. In exemplary embodiments, channel 110 comprises a liquid and a polyacrylamide gel. In the illustrated embodiment, first reservoir 101, second reservoir 102, and third reservoir 103 contain first electrolyte solution (also referred to herein as a leading electrolyte solution) and fourth reservoir 111 contains a second electrolyte solution (also referred to herein as a trailing electrolyte solution). It is understood that other exemplary embodiments may comprise a different number of reservoirs containing the first and/or second electrolyte solutions than the embodiment shown in FIG. 2A.

In the embodiment shown, the polyacrylamide gel in channel 110 varies in concentration in the liquid between reservoir 103 (containing the first or leading electrolyte solution) and reservoir 111 (containing the second or trailing electrolyte solution). In specific embodiments, channel 110 contains a cell lysate between reservoir 111 and reservoir 101. In the illustrated embodiment, channel 110 also contains a first concentration of polyacrylamide gel between reservoir 101 and reservoir 102, and channel 110 also contains a second concentration of polyacrylamide gel between reservoir 103 and 102. In exemplary embodiments, the second concentration of polyacrylamide gel is greater than the first concentration of polyacrylamide gel. In specific embodiments, the first concentration of polyacrylamide gel is 2, 3, 4, 5, 6 or 7 percent and the second concentration of polyacrylamide gel is 8, 9, 10, 11 or 12 percent.

In this embodiment, apparatus 100 also comprises a power supply 150 coupled to a first electrode 151 and a second electrode 152. In the illustrated embodiment, first electrode 151 is located in (or in electrical communication with) reservoir 103 and second electrode 152 is located in (or in electrical communication with) reservoir 111. Exemplary embodiments may comprise additional electrodes (not illustrated for purposes of clarity) for example, in reservoir 101 and reservoir 102.

In particular embodiments, power supply 140 can apply voltage differential between first electrode 151 and second electrode 152 (and additional electrodes if so equipped) such that a current is applied to channel 110. In certain embodiments, a control circuit 150 can control the operation of power supply 140 and the current and/or voltage applied to channel 110. As described elsewhere in the present disclosure, apparatus 100 can be operated to extract RNA in the size range of 20-35 nt from cell lysate in channel 110.

### Example 2—Ribo-ITP Enables High Quality Single Cell Ribosome Occupancy Measurements

A critical factor in generating ribosome profiling data is the choice of the ribonuclease (Reid et al., 2015). Here, micrococcal nuclease (MNase) was employed to maximize the recovery of ribosome protected RNA fragments (Paris et al., 2019; Vu et al., 2017; Zhao et al., 2019) at the expense of a slight loss in nucleotide resolution, which was able to be ameliorated by computational means (Wolin & Walter, 1988). To validate the quality of ribosome profiling data, Ribo-ITP was first performed from 100 K562 cells and conventional ribosome profiling was performed according to the gold-standard method of monosome isolation16 from 10 million K562 cells. For sequencing library preparation, a single tube protocol was optimized that incorporated unique molecular identifiers (UMIs) via a template switching reverse transcriptase (FIG. 6C).

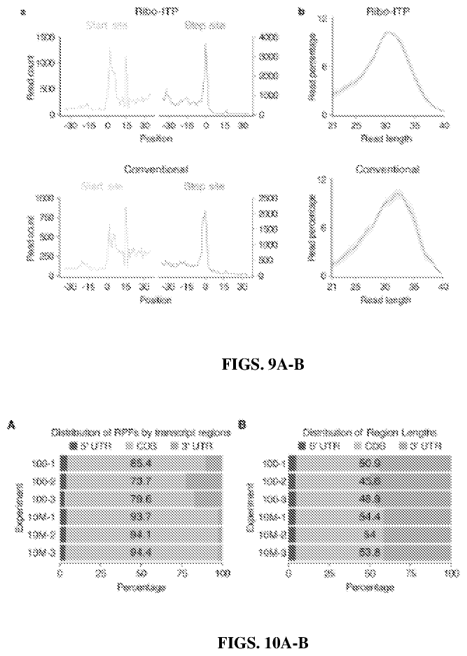

Ribosome occupancy measurements from 100 cells obtained using Ribo-ITP were highly reproducible across replicates (FIG. 6D; FIG. 8; Supplementary Table S1). The footprints displayed the characteristic read length distribution (Chen et al., 2011) and the expected enrichments at annotated translation start and stop sites (FIG. 9). The vast majority of transcript mapping reads originated from the coding regions and were highly enriched over the distribution expected from random fragmentation (Chi-squared test, p-value <2.2×10⁻¹⁶; FIG. 10). Critically, ribosome profiling measurements from 100 cells generated by Ribo-ITP recapitulated the conventional ribosome profiling measurement (Spearman correlation coefficient 0.88; p-value <2.2×10⁻¹⁶, FIG. 6D). These results revealed that ribosome occupancy was able to be accurately measured from as few as 100 human cells using Ribo-ITP.

TABLE S1
Ribosome Occupancy Measurements

| | | | | | | Usable |
|---|---|---|---|---|---|---|
| | | | | | | Reads |
| | | | | non | | After |
| | Total | Clipped | rRNA & | rRNA & | Transcriptome | UMI |
| | Reads | Reads | tRNA | tRNA | Aligned Once | Dedup |
| 20210301- | 29378123 | 25893337 | 14179874 | 11713463 | 541133 | 203263 |
| ITP-MII- | | | | | | |
| 25-B | | | | | | |
| 20210301- | 24675567 | 21891106 | 10637224 | 11253882 | 619005 | 233925 |
| ITP-MII- | | | | | | |
| 50-A | | | | | | |
| 20210301- | 28911387 | 26278109 | 13470440 | 12807669 | 674088 | 240765 |
| ITP-MII- | | | | | | |
| 50-B | | | | | | |
| 20210318- | 53190110 | 45439127 | 24973290 | 20465837 | 1700978 | 375367 |
| ITP-MII- | | | | | | |
| 50-B | | | | | | |
| 20210614- | 33476569 | 29175709 | 14454091 | 14721618 | 1563076 | 317673 |
| ITP-MII- | | | | | | |
| 50-D | | | | | | |
| 20210513- | 37085059 | 29942481 | 14940202 | 15002279 | 1417438 | 364866 |
| ITP-1cell- | | | | | | |
| cross-50- | | | | | | |
| A | | | | | | |
| 20210513- | 60469037 | 48590009 | 21888576 | 26701433 | 2126755 | 353570 |
| ITP-1cell- | | | | | | |
| cross-50- | | | | | | |
| B | | | | | | |
| 20210513- | 32160471 | 26208079 | 14122428 | 12085651 | 867763 | 268720 |
| ITP-1cell- | | | | | | |
| cross-50- | | | | | | |
| C | | | | | | |
| 20210513- | 33676015 | 27856960 | 15191367 | 12665593 | 1305570 | 339588 |
| ITP-1cell- | | | | | | |
| cross-50- | | | | | | |
| D | | | | | | |
| 20210513- | 46685730 | 38724676 | 19778281 | 18946395 | 1954290 | 457278 |
| ITP-1cell- | | | | | | |
| cross-50- | | | | | | |
| E | | | | | | |
| 20210513- | 61716791 | 54625475 | 23471055 | 31154420 | 1543305 | 374338 |
| ITP-2cell- | | | | | | |
| cross-50- | | | | | | |
| B | | | | | | |
| 20210513- | 46359612 | 39480188 | 22838899 | 16641289 | 1203693 | 270424 |
| ITP-2cell- | | | | | | |
| cross-50- | | | | | | |
| C | | | | | | |
| 20210513- | 46129676 | 37831229 | 15768657 | 22062572 | 1547470 | 354498 |
| ITP-2cell- | | | | | | |
| cross-50- | | | | | | |
| F | | | | | | |
| 20210513- | 55110601 | 43640368 | 10614145 | 33026223 | 2695752 | 902628 |
| ITP-4cell- | | | | | | |
| cross-50- | | | | | | |
| B | | | | | | |
| 20210513- | 56432637 | 47173137 | 27798471 | 19374666 | 1252463 | 441544 |
| ITP-4cell- | | | | | | |
| cross-50- | | | | | | |
| C | | | | | | |
| 20210513- | 42127261 | 35047820 | 13665662 | 21382158 | 1742092 | 371102 |
| ITP-4cell- | | | | | | |
| cross-50- | | | | | | |
| D | | | | | | |
| 20210513- | 23754234 | 18970212 | 7722146 | 11248066 | 903033 | 283914 |
| ITP-8cell- | | | | | | |
| cross-50- | | | | | | |
| A | | | | | | |
| 20210513- | 53302424 | 44964420 | 23450782 | 21513638 | 1875252 | 523253 |
| ITP-8cell- | | | | | | |
| cross-50- | | | | | | |
| B | | | | | | |
| 20210513- | 24020697 | 19216806 | 7282520 | 11934286 | 948894 | 370678 |
| ITP-8cell- | | | | | | |
| cross-50- | | | | | | |
| C | | | | | | |
| 20210513- | 35039279 | 28734732 | 11405020 | 17329712 | 1031266 | 336858 |
| ITP-8cell- | | | | | | |
| cross-50- | | | | | | |
| D | | | | | | |
| 20210614- | 17255218 | 13935949 | 5147806 | 8788143 | 1624661 | 382706 |
| ITP-GV- | | | | | | |
| 50-A | | | | | | |
| 20210614- | 29023853 | 23524609 | 7047450 | 16477159 | 1926792 | 443062 |
| ITP-GV- | | | | | | |
| 50-B | | | | | | |
| 20210614- | 31546023 | 25997628 | 7102255 | 18895373 | 2190082 | 677341 |
| ITP-GV- | | | | | | |
| 50-C | | | | | | |
| 20210614- | 20694464 | 16981727 | 6202918 | 10778809 | 1780939 | 586192 |
| ITP-GV- | | | | | | |
| 50-E | | | | | | |
| 20210614- | 17978166 | 15345654 | 4808388 | 10537266 | 1153496 | 427053 |
| ITP-GV- | | | | | | |
| 50-F | | | | | | |

Next, Ribo-ITP and RNA-seq was applied to characterize the translation changes of single cell and single embryos in the context of early embryonic development in mice. In particular, the initial division of zygotes occurs in the absence of new RNA synthesis, rendering the translation of stored maternal transcripts is relevant for the early stages of development. Hence, Ribo-ITP and RNA-seq were used to analyze multiple stages of preimplantation development including single unfertilized oocytes at germinal vesicle (GV) and metaphase II (MII) stages, as well as single fertilized embryos from the 1-cell zygote to 8-cell stages (FIG. 11A; Table S1).

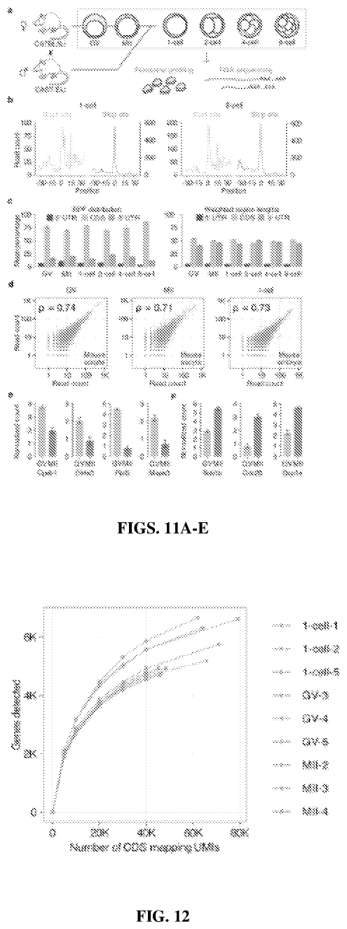

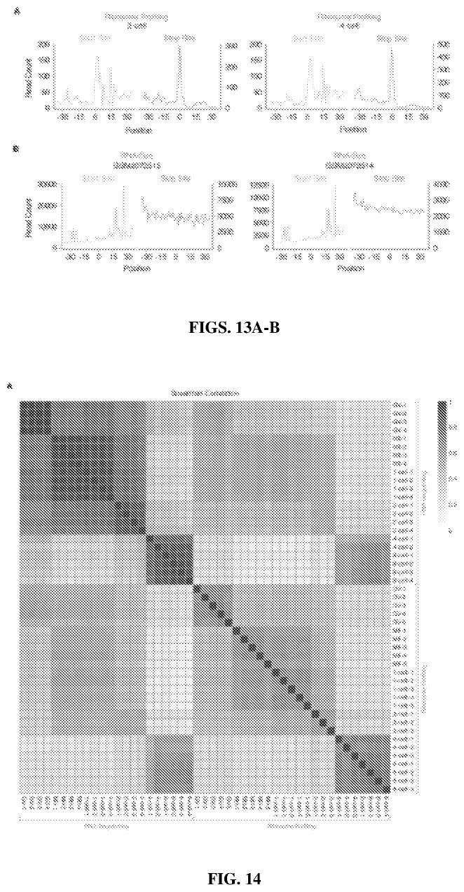

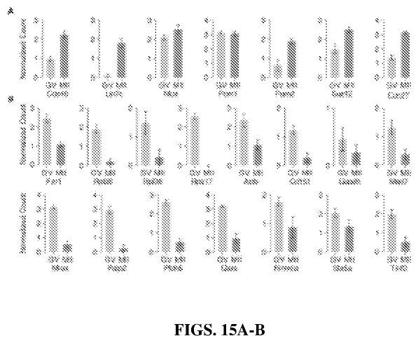

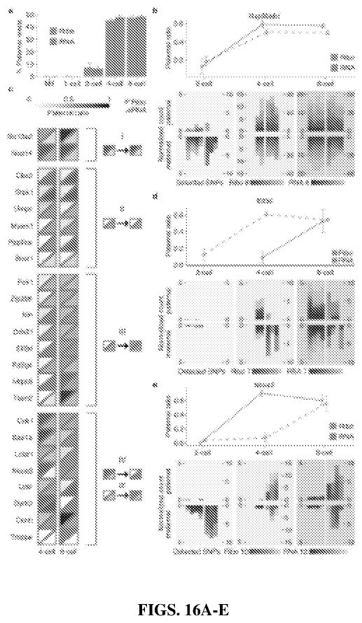

In the single cell ribosome occupancy data, a median of 48,017 unique molecules were observed to originate from the coding regions of transcripts, which resulted in detection of an average of 5064 genes per cell (range 4076-6679; FIG. 12). Furthermore, single oocyte and single embryo ribosome profiling data demonstrated the expected enrichment of footprints mapping to coding regions and characteristic enrichments at the start and stop sites (FIG. 11B-C, FIG. 13). Replicate measurements of ribosome occupancy had high correlation with each other (FIG. 11D; FIG. 14).

To validate the quality of the single cell ribosome profiling measurements, the results were compared to a complementary approach for assaying translation; polysome profiling, a strategy that can inform the distribution of the number of ribosomes on a given mRNA. In particular, a previous study collected ˜500-600 GV- and MII-stage oocytes and validated changes in polysome association with rt-qPCR experiments for 29 transcripts (Deng et al., 2014). Remarkably, the single cell ribosome profiling measurements of the invention recapitulated the previously identified changes in polysome association for 28 out of 29 RNAs (FIG. 11E, FIG. 15). Taken together, the results indicated that Ribo-ITP enabled highly consistent and high quality ribosome occupancy measurements from single cells and single embryos during early mouse development.

### Example 3—Ribo-ITP Reveals Allele-Specific Translation in Mouse Preimplantation Development

In mouse development, transcription of the zygotic genome is activated at the 2-cell stage. Yet, it is not currently known when newly synthesized RNAs engage with ribosomes and whether there exist any gene- and allele-specific differences in these dynamics. The question of allele-specific expression following zygotic genome activation was initially addressed. Without being bound by theory, both deterministic and stochastic differences in allele expression ratios are believed to contribute to differentiation and normal development, though studies had been limited to the level of epigenetics and transcription in the early mouse embryo25, 26. Since these studies typically require single-cell resolution, it has been impossible to study allele-specific translation until now.

To distinguish RNAs derived from the maternal and paternal alleles, embryos from a cross of two mouse strains (C57BL/6J×CAST/EiJ) were analyzed. Using strain-specific single-nucleotide polymorphisms (SNPs) to distinguish maternal and paternal RNAs, 229,991 unique parent-of-origin-specific RPFs mapping to coding regions were detected. (Example 6). As a control for the accuracy in alignments and SNP annotation, unfertilized MII-stage oocytes were analyzed and found 97.3% correctly classified reads, i.e. reads with maternal SNPs, in the ribosome profiling experiments described herein.

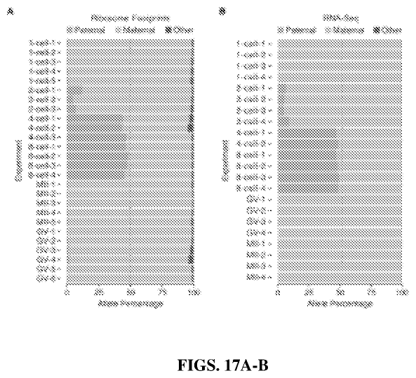

To monitor allele-specific ribosome engagement alongside corresponding RNA expression (Nagaoka et al., 2012), the paternal allele, which unlike RNA of maternal origin is a proxy of newly synthesized transcripts, was investigated. (Example 6). The global pattern of ribosome engagement of paternally-derived RNAs, i.e., paternal allele ratios, was analyzed by aggregating reads across all detected genes. It was found that, coinciding with the activation of zygotic transcription, the paternal ratio of ribosome occupancy steadily increased from 7.1% in the 2-cell stage to 47.7% in the 8-cell stage embryos (FIG. 16A; FIG. 17). Importantly, the ratio of paternal alleles across these stages was discovered to be statistically indistinguishable between ribosome occupancy and RNA expression (t-test; p-value>0.14 for all stages; FIG. 16A). This result indicated that ribosome engagement was overall concurrent with the synthesis of paternal RNAs via zygotic genome activation.

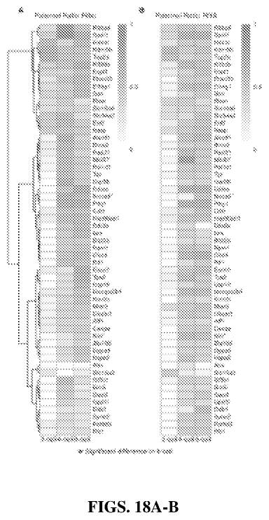

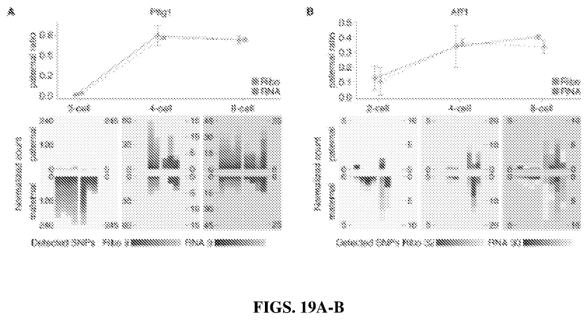

Next, the existence any gene-specific exceptions to the observed global pattern of equal paternal allelic ratios in RNA expression and ribosome occupancy in the early mouse embryo was considered. The high coverage data achieved by the inventors enabled assessment of these dynamics in 1012 genes (Example 6). The majority of genes exhibited a similar ratio of paternal reads in both RNA expression and ribosome occupancy (FIG. 18). For example, 8 distinct coding SNPs were detected that differentiated the two alleles in Hsp90ab1 across multiple replicates in RNA-seq and Ribo-ITP. The high similarity of paternal allele ratio in RNA expression and ribosome occupancy was consistently observed for multiple replicates and supported by distinct SNPs, highlighting the reproducibility and high coverage of these allele-specific measurements (FIG. 16B, FIG. 19).

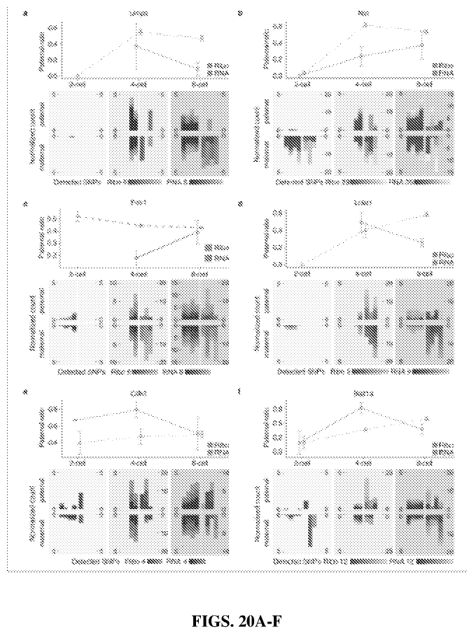

24 genes were identified that had differential ribosome engagement in an allele-specific manner in comparison to RNA expression (two-sample test for the equality of proportions; see Example 6 for details; FIG. 16B). Four clusters were identified among these 24 genes (FIG. 16C). While clusters I and II encompassed genes that displayed consistent allele-specific ribosome occupancy bias throughout early development (FIG. 20A), genes in the other two clusters display allele-specific ribosome occupancy in a stage-dependent manner.

In particular, several genes including Eif3d display delayed engagement of newly transcribed paternal RNA with ribosomes. Specifically, paternal allele was robustly expressed in 4-cell embryos, yet ribosome association of the paternal allele is delayed until the 8-cell stage (FIG. 16E; Cluster III, FIGS. 20B-C). Without being bound by theory, this observation suggested that specific transcripts may either have had slow kinetics of nuclear export or were sequestered in translationally inactive compartments until their subsequent association with ribosomes occurred in the 8-cell stage.

Genes in the last group (Cluster IV) included Cdk1, a key regulator of cell cycle, Baz1a, a chromatin remodeling factor, and Lclat1, lysocardiolipin acyltransferase 1 (FIG. 16D; FIGS. 20D-F). While some of these genes had differential ribosome occupancy of the paternal allele compared to RNA expression in only the 4-cell stage (e.g. Baz1a), others (e.g. Lclat1) differed at the 8-cell stage. Without being bound by theory, these findings suggested the presence of an interaction between the cis-elements and regulatory factors that enabled differential ribosome occupancy of one of the alleles only in a specific stage of development. Taken together, the results revealed that for most transcripts, ribosome engagement was concurrent with zygotic activation and paternal RNA expression. However, a small number exhibit allele-specific ribosome engagement included stage-specific differences.

### Example 4—Differential Translation Efficiency During Oocyte Maturation and Preimplantation Development Regulates a Functionally Coherent Set of Genes

The proteome of the zygote is composed of maternally deposited proteins and those newly synthesized after fertilization (Vastenhouw et al., 2019). However, it has not been possible to study the relative contribution of maternally deposited versus newly synthesized proteins to the zygotic proteome at a transcriptome-wide scale in a mammalian system. Ribo-ITP was applied to assess the contribution of translation in determining protein abundance (Gao et al., 2017). In particular, protein abundance of 3,287 out of >5,000 transcripts detected in the single embryo ribosome profiling and RNA-Seq method of the inventors had previously been quantified using mass spectrometry of ˜8000 embryos from each stage of mouse preimplantation development (Gao et al., 2017).

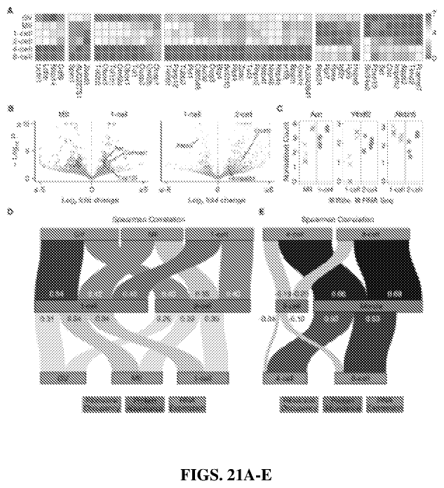

The zygotic proteome was found to be only modestly correlated with RNA expression of the zygote (Spearman Rank Correlation 0.34; p-value<2.2×10⁻¹⁶); in agreement with previous work that reported weak correlation between RNA expression and protein abundance (Gao et al., 2017; Tang et al., 2009). In contrast, zygotic protein abundance was significantly better correlated with ribosome occupancy than RNA expression of the zygote (Spearman Rank Correlation 0.45 vs 0.34; p-value<2.2×10⁻¹⁶; FIG. 21D).

It was discovered that ribosome occupancy of the GV-stage oocytes had the strongest relationship with the zygotic protein abundance (FIG. 21D; Spearman Rank Correlation 0.54, p-value<2.2×10⁻¹⁶). Importantly, this key contribution was undetectable at the level of RNA expression as RNA abundance in GV-stage oocytes is much more weakly associated with the zygotic protein abundance (Spearman Rank Correlation 0.31; p-value<2.2×10⁻¹⁶). Taken together, these results revealed that maternal translation is the predominant contributor to the zygotic proteome; a finding that eluded RNA-seq analyses and, without being bound by theory, resolves the apparent paradox of low correlation between RNA and protein abundance at this critical stage in development.

The coupling of rapid degradation of the maternally deposited RNAs and onset of zygotic transcription fundamentally remodels the RNA contents of the developing embryo. Consequently, the 4-cell stage embryos have a very different RNA composition compared to 1-cell and 2-cell stage embryos. Intriguingly, neither ribosome occupancy nor RNA expression was positively correlated with protein abundance at the 4-cell or the 8-cell stage (FIG. 21E). Instead, ribosome occupancy at the 4-cell and 8-cell stage embryos was found to be much more strongly associated with the protein abundance at the morula stage. The association with protein abundance was significantly stronger for ribosome occupancy compared to RNA expression (FIG. 21E; Spearman Rank Correlation 0.66 vs 0.50 at 4-cell stage and 0.69 vs 0.53 at 8-cell stage, p-value<2.2×10⁻¹⁶). These results suggested an unanticipated role of ribosome occupancy of the 4-cell and 8-cell stage embryos in determining morula protein abundance.

### Example 5—Interpretation of Experimental Results

The ribosome profiling described herein allowed deciphering of the translational landscape at transcriptome-wide scale. Conventional ribosome profiling approaches involve multiple steps with significant loss of input and require considerable amounts of starting material. Consequently, many biological questions of importance have been beyond the scope of the conventional ribosome profiling in the last decade. Here, a method, RIBOsome profiling via IsoTachoPhoresis (Ribo-ITP), was presented to overcome this limitation. The approach of the inventors enabled the study of translation in precious samples with limited input amounts such as human biopsies, embryonic tissues, cancer stem cells and transient populations.

Ribo-ITP involved the use of a microfluidic chip that couples ITP-based RNA extraction with on-chip precise size-selection of ribosome footprints. The most prominent advantage of ITP-based extraction in contrast to conventional liquid- or solid-phase extractions was the high yield regardless of input amount or nucleic acid size distribution (Han et al., 2019; Buenrostro et al., 2015; Schier et al., 2020). The most stringent RNA size selection with the highest yield from materials as little as a single cell was achieved among all previously described RNA extraction and size selection methods using ITP (Han et al., 2019; Eid & Santiago, 2017). In addition, 3′ ddC blocking modification and the optimized buffer exchange protocol broadened the downstream applicability of ITP-based sample purification to a variety of next generation sequencing-based analyses (Marshall et al., 2014; Kuriyama et al., 2015). Ribo-ITP was applied to study early stages of mouse embryonic development, where translational control of gene expression plays an imperative role (Vastenhouw et al., 2019; VanInsberghe et al., 2021). The resultant high coverage data enabled the first analysis of allele-specific ribosome occupancy during preimplantation development. The allele-specific ribosome occupancy and RNA expression of more than one thousand genes from single oocytes and embryos was characterized. Translation of nascent transcripts after zygotic gene activation was shown for the first time to be delayed until the 2-cell stage (Abdelmeoz et al., 2018). Reduced ribosome association with the parental allele until the 2-cell stage was observed. While cis-regulation driven allele-specific effects were known in other systems, the study described herein revealed two additional patterns. Interestingly, a cluster of genes was identified which showed allele-specific ribosome occupancy in a stage-specific manner. Without being bound by theory, this could potentially be due to the stage-specific expression of specific translation activators or degradation of repressors. Though most of the genes had concurrent RNA expression and ribosome occupancy, the fourth cluster of genes in particular showed a lag between the two parameters. Possible reason for this observation, without being bound by theory, may be differences in ribosome loading or delayed nucleus to cytosol transport of mRNA relevance (Khmouf et al., 2018; Evsikov et al., 2009).

Preimplantation development of mouse embryos requires a precise proteome profile which is governed by its transcriptomic landscape. However, correlation between these two parameters within a stage is poor (Chen et al., 2017). Here, it was shown that ribosome occupancy was a significantly better predictor of protein abundance and more strikingly the strongest relationship between ribosome occupancy and protein abundance was established with a time lag. The ribosome occupancy profile of the GV-stage of the oocyte correlated best with the zygotic proteome. Similarly, it was discovered that ribosome occupancy of transcripts in 4- and the 8-cell set the stage for the proteome of the morula. A missing link to the genotype-phenotype relationship was provided and new avenues were opened for exploring these dynamics in other biological systems, particularly rare cell populations.

### Example 6—Experimental Methods

### Polydimethylsiloxane (PDMS) Chip Fabrication

Molds were 3D-printed by Proto Labs with WaterShed XC 11122 at high resolution (FIG. S1). Reusable molds were assembled by taping 3D-printed molds to glass slides (5″ by 4″; Ted Pella). Sylgard 184 PDMS monomer and curing agent (Ellsworth Adhesives 4019862) were mixed at a 10:1 (w/w) ratio. The mixture was degassed using a dessicator connected to a vacuum pump, poured over the mold, and degassed again until there were no air bubbles. The mold was incubated for at least 16 h at 50° C. Individual PDMS chips were cut along the lines that form the outer rectangle on the design in FIG. S1A. The 5 mm-diameter elution well, TE-, and LE-reservoirs were made with a biopsy punch (FIG. 2B). Prior to the plasma treatment, glass slides (4″ by 3″; Ted Pella) and the feature-side of the PDMS slabs were thoroughly cleaned with tape to remove any dust particles. PDMS chips and glass slides were plasma cleaned with a 115V Expanded Plasma Cleaner (Harrick Plasma) connected to a Dry Scroll Pump (Agilent) for 2 minutes at high RF level. The plasma-treated surfaces of the glass and PDMS slabs were immediately brought together to form a covalent bond. Bonded chips were heated at 80° C. on a heat block for at least two hours to enhance bonding.

### PDMS Chip Preparation for Ribo-ITP Experiments

To ensure clean, RNAse-free chips, the channels and reservoirs of the Ribo-ITP chip were pre-treated by sequential treatment with the following solutions: RNaseZap (100% concentrate), nuclease-free water, 1 M NaOH, nuclease-free water, 1 M HCl, nuclease-free water, 10% (w/v) benzophenone in acetone (for 10 minutes, replenishing channels as needed to avoid bubble accumulation), methanol, and 0.1% Triton X-100. The channel was completely dried after final treatment by fully vacuuming out any remaining liquid in the channel. After securing the chips to a ProteinSimple 302/365 nm UV Transilluminator with tape, 10% polyacrylamide (PA) prepolymer mix (Table 2) to the size-selection channel was added through the elution well. Similarly, 5% PA prepolymer mix was loaded into the extraction channel through branch channel 2. To catalyze the polymerization of polyacrylamide on chip, a photoactivatable azo-initiator, 2,2′-Azobis [2-methyl-N-(2-hydroxyethyl) propionamide] (Wako Chemicals VA-086), was used at 0.5% final concentration in the prepolymer mixes. UV-driven polymerization (365 nm wavelength) was performed for one minute followed by a 30 second break. This on/off UV cycle was repeated two more times for a total UV exposure time of 3 minutes. UV intensity was measured as ˜8.9 mW/cm2 using a G&R Labs Model 200 UV Light meter with a 365 nm probe. To avoid dehydration of the polyacrylamide gels after polymerization, any open channels and reservoirs were filled with storage buffer (Table 2) until use. The chips were protected from light and used within six hours of preparation. 1-(1-methyl-1H-pyrrol-2-yl) ethanone (35.4 g, 0.157 mol) was dissolved in acetic anhydride (200 mL). Obtained solution was cooled down to −40° C. Nitric acid (d=1.5 g/mL, 1.8 eq, 14 mL) was added over period of 30 minutes. The reaction mixture was allowed to warm-up to room temperature, and stirring was continued for additional 2 hrs. The mixture was cooled down to −20° C., then isopropyl alcohol was added. (H-NMR (δ, CDCl₃, ppm) 7.91 (bs, 1H, H-5), 7.76 (bs, 1H, H-3), 4.04 (s, 3H, Me)).

TABLE 2
Buffer preparation for on-chip isotachophoresis

| Name | Contents | Notes |
|---|---|---|
| 1M BisTris HCl pH 7.2 | 1M BisTris titrated to pH | Check the pH of an |
| | 7.2with HCl | aliquotwith a pH meter |
| | | (should be ~7.2 +/− ~0.02). |
| 2.5M BisTris | 2.5M BisTris | Prepare in advance and |
| | | storeat 4° C. |
| BisTris HCl Master Mix | 2.5M BisTris titrated to pH | Check the pH of an |
| | 7.2 with HCl | aliquotwith a pH meter |
| | | (should be ~7.2 +/− ~0.02). |
| Lysis buffer stock | 20 mM BisTris (titrated, pH | Prepare in advance and |
| | 7.2), 1.0% Triton-X100, 5 mM | store |
| | MgCl₂, 5 mM CaCl₂, 100 | at 4° C. |
| | mM NaCl | |
| Lysis buffer - working solution | 20 mM BisTris (titrated, pH | Prepare fresh for each |
| | 7.2), 1.0% Triton-X100, 5 | experiment, keep on ice. |
| | mM MgCl₂, 5 mM CaCl₂, | Cycloheximide should |
| | 100 mM NaCl, 100 μg/mL | be handled in a |
| | cycloheximide, 1 mM DTT | chemical hood. |
| Lysis buffer (for | 20 mM Tris HCl pH 7.4, 150 | |
| ribosomeprofiling by | mM NaCl, 1% Triton-X 100, 5 | |
| monosome isolation) | mM CaCl₂, 5 mM MgCl₂, 25 | |
| | U/mL Turbo DNase I, 100 | |
| | μg/mL Cycloheximide, 1 | |
| | mM DTT and 1X Protease | |
| | Inhibitor Cocktail (EDTA- | |
| | free) | |
| Leading electrolyte | 25% Pluronic F-127, 50 mM | Prepare fresh, ideally |
| pluronicsolution (LEp) | HCl, 200 mM BisTris, and | before each ITP |
| | nuclease-free water to 25 mL | experiment. For LEp, |
| Trailing electrolyte | 25% Pluronic F-127; 100 mM | best practice is to makea |
| pluronicsolution (TEp) | MOPS; 200 mM BisTris, and | master mix of BisTris |
| | nuclease-free water to 25 mL | HCl and check the pH of |
| | | an aliquot with a pH |
| | | meter (should be ~7.2 +/− ~0.02). |
| | | Add chemical |
| | | components to a conical |
| | | tube containing the |
| | | pluronic F-127 with ~10 |
| | | ml nuclease-free H₂O. Let |
| | | spin overnight at 4° C. as |
| | | pluronic F-127 is soluble |
| | | in water at cold |
| | | temperatures. Spin down |
| | | solutions at 4° C. and |
| | | bring the volume to 25 |
| | | mL with nuclease-free |
| | | water. Keep at 4° C. if |
| | | storing and keep on ice |
| | | during ITP experiments. |
| | | Pipet with frozen tips to |
| | | avoidpremature |
| | | solidification. |
| 5% polyacrylamide | 5% polyacrylamide, 130 mM | To dissolve urea, it is |
| (PA)prepolymer mix | BisTris, 20 mM HCl, 8M | necessary to combine |
| | urea, 0.5% 2,2′-Azobis[2- | all components besides |
| | methyl-N-(2- | 2,2′- |
| | hydroxyethyl)propionamide] | Azobis[2-methyl-N-(2- |
| 10% polyacrylamide | 10% polyacrylamide, 130 mM | hydroxyethyl)propionamide]and |
| (PA)prepolymer mix | BisTris, 20 mM HCl, 8M | heat (recommended |
| | urea, 0.5% 2,2′-Azobis[2- | 50° C. on a |
| | methyl-N-(2- | heat block) and |
| | hydroxyethyl)propionamide] | vortex until |
| | | homogenized. Add 2,2′- |
| | | Azobis[2-methyl-N-(2- |
| | | hydroxyethyl)propionamide], |
| | | vortex to |
| | | homogenize, and |
| | | protect the solution |
| | | from light. |
| Running buffer (RB) | 130 mM BisTris, 20 mM HCl, | |
| | 0.1% PVP | |
| Sample dilution buffer (SDB) | 10.667M Urea, 2.67% PVP, | Prepare fresh the day of |
| | 130 mM BisTris, 20 mM HCl | theexperiment. |
| LE Storage buffer | 130 mM BisTris, 20 mM HCl, | |
| | 0.5% PVP | |
| 10% (w/v) benzophenone in | 10% (w/v) benzophenone in | Prepare fresh the day of |
| acetone | acetone | theexperiment. |
| 2X denaturing gel loading dye | 89 mM Tris; 89 mM Boric | Prepare in advance and |
| | acid; 2 mM EDTA, pH 8.0; | storesingle use aliquots |
| | 12% Ficoll; 0.01% | in −20° C. |
| | Bromophenol Blue; 7M | |
| | urea | |
| 1X TBE buffer | 89 mM tris, 89 mM | Purchased as a |
| | borate, and 2 mM EDTA | 10Xconcentrate |
| Gel extraction buffer | 300 mM sodium acetate (pH | Prepare in advance and |
| | 5.2), 1 mM EDTA, 0.25% | storeindefinitely at room |
| | (wt/vol) SDS | temperature |
| Dephosphorylation Buffer | 8:2 ratio of nuclease-free | Dephosphorylation |
| | water and Dephosphorylation | Buffer(DB) supplied |
| | Buffer (Diagenode) | with D-PlexSmall |
| | | RNA-seq Kit |
| | | (Diagenode |
| | | C05030001) |

### Isotachophoresis Setup

The prepared ITP chip was placed on a Dark Reader blue light transilluminator (Clare Chemical) and secured with tape. Storage buffer was removed from the channels and reservoirs using a vacuum. Leading electrolyte pluronic solution (LEp) and MOPS trailing electrolyte pluronic solution (TEp) (Table 2) were kept on ice throughout the loading procedure. 200 μL pipet tips were kept at −20° C. until the time of the experiment to facilitate manipulation of the pluroniccontaining LEp and TEp solutions, which solidify within a minute above 4° C. 80 μL of LEp was loaded in LE reservoir 3, filling the reservoir to the top as well as the small section of the channel between the elution well and LE reservoir 3 (FIG. 2B). LE reservoir 2 was filled with 30 μL LEp, ensuring contact with the polyacrylamide gel present in branch channel 2. The elution well was filled with 20 μL of RB. Fluorescent marker oligonucleotides containing a 5′ ATTO fluorophore and 3′ ddC blocking modification (Table 3) were added to the sample followed by dilution with sample dilution buffer (SDB). The mixture was loaded into the lysate channel through LE reservoir 1. Finally, LE reservoir 1 was filled with 30 μL LEp and 70 μl Tep was added to the TE reservoir. The negative electrode was placed in the TE reservoir and the positive electrode in the LE reservoir. Positive and negative electrodes were placed in LE reservoir 3 and the TE reservoir, respectively. A constant current of 300 mA with a maximum voltage of 1.1 kV (Keithley 2410 Sourcemeter) was applied to the channel. Once the trailing end of the fluorescent markers entered the 5% PA gel, the branch channel electrode—with a lower current output due to a 510 kΩ (Xikon) resistor on a custom circuit board—was manually applied in LE reservoir 1 for ˜10 seconds. When the leading edge of the longer fluorescent marker reached the end of the size-selection channel, the current was suspended. The elution reservoir was thoroughly washed twice with 30 μL nuclease-free water and refilled with 10 μL dephosphorylation buffer (Table 2). Current was applied again until the shorter fluorescent marker began to enter the elution well. Finally, the purified sample with a μL volume was collected from the elution well into a low-bind PCR tube and immediately stored at −80° C.

A solution of 2,2,2-trichloro-1-(1-methyl-4-nitro-1H-pyrrol-2-yl) ethanone (24 g, 87 mmol) in methanol (75 mL) was added dropwise to the suspension of NaH (300 mg) in methanol (30 mL). The reaction mixture was stirred at room temperature for 2 hrs, then the reaction was quenched by addition of concentrated sulfuric acid (0.75 mL). The reaction mixture was then heated to reflux and allowed to slowly cool to room temperature. Product crystallized from reaction mixture as white needles. Product was filtered and dried under vacuum. (H-NMR (δ, DMSO-d₆, ppm) 8.27 (bs, 1H, H-5), 7.31 (bs, 1H, H-3), 3.93, 3.80 (2s, 3H ea, Me))

TABLE 3
Oligonucleotides

| | | | SEQ | |
|---|---|---|---|---|
| | | | ID | |
| Name | Supplier | Sequence | NO: | Notes |
| 19 nt | IDT | /5ATTO488N/CTGCTGGAGTTC | 4 | Purchased with |
| fluorescent | | GTGACC/3ddC/ | | dual HPLC |
| DNA | | | | purification, then |
| marker | | | | gel purifiedin-lab |
| | | | | to improve purity |
| 36 nt | IDT | /5ATTO488N/CCTCCTTGTATAA | 5 | |
| fluorescent | | ATCCTGGTTGCTGTCTCTTTAT/ | | |
| DNA | | 3ddC/ | | |
| marker | | | | |
| 17 nt RNA | IDT | 5′- | 6 | |
| marker | | /rArUrCrArGrGrGrCrCrArUrArArC | | |
| | | rArG/3ddC/ | | |
| 26 nt RNA | BSI | 5′- | 7 | |
| marker | | /rArUrGrUrUrArGrGrGrArUrArAr | | |
| | | CrArGrGrGrUrArArUrGrCrGrA/3 | | |
| | | [Phos] | | |
| 34 nt RNA | BSI | 5′- | | |
| marker | | /rArUrGrUrArCrArCrUrArGrGrGr | 8 | |
| | | ArUrArArCrArGrGrGrUrArArUrCr | | |
| | | ArArCrGrCrGrA/3[Phos] | | |

### Polyacrylamide Gel Electrophoresis and Conventional Extraction of RNA

Control inputs were prepared as a master mix then aliquoted. For gel extraction samples, input RNA was first processed using Qiagen miRNeasy Micro Kit per manufacturer's instructions. RNAs were separated by electrophoresis using 15% TBE-Urea polyacrylamide gel (Invitrogen EC6885BOX). Gel slices were excised and crushed using sterile pestles, followed by soaking in gel extraction buffer (Table 2) on dry ice for 30 minutes. Samples were then incubated overnight at room temperature, gently transferred on a tabletop shaker and protected from light. Residual gel pieces were removed by centrifugation for 1 minute at 21,130×g through a Corning 0.22 μm sterile filter tube. The recovered eluate was precipitated overnight at −20° C. (300 mM sodium acetate pH 5.2, 5 mM MgCl2, 1.5 μL Glycoblue, 75% ethanol). Samples were pelleted by centrifugation at 4° C. for 1 hr at 21,130×g.

### Gel Imaging and Quantification

To quantify yield, samples were run on a 15% TBE-Urea polyacrylamide gel and visualized using the fluorescent marker oligonucleotides or by SYBR Gold-staining. Specifically, gels were imaged using Typhoon FLA 9500 (GE Healthcare) with a 473 nm excitation wavelength and LPB filter compatible with ATTO488 fluorophore and SYBR gold-stain. For high resolution imaging, pixel size was minimized (10-25 μm) and PMT settings were optimized by using Typhoon's scanning feature to avoid image oversaturation; typically resulting in a PMT value between 250-500 V. The images were analyzed using ImageJ software (NIH). Raw integrated density (RID) for background signal (RID_{background}) was measured by quantifying average RIDs from representative blank areas. RID_{background} Was normalized to account for the ratio of the target (Aₛₐₘₚₗₑ) to the background area (A_{background}) such that

$${background}_{normalized} = {RID}_{background} * ( {A}_{sample} ) / ( {A}_{background} )$$

The normalized background value was subtracted from all samples to quantify normalized sample RID values. The percent yield was defined as the ratio of the normalized RID values to the mean of background-normalized input samples. For display purposes only, the contrast and brightness of some images were adjusted in ImageJ and exported as tiff files for figures.

### Yield Comparison Between On-Chip Method and Conventional RNA Extraction

Input controls and experimental samples were prepared with a final total amount of 40 ng, 20 ng, 2 ng, 400 pg, or 40 pg of ZR small RNA ladder (Zymo Research R1090) including 17, 21, 25, and 29 nt RNA oligonucleotides. Ribo-ITP was performed as described, with a final elution in 12 μL RB. Samples for gel extraction were first processed with the miRNeasy micro kit (Qiagen), followed by extraction using the crush & soak approach. Only the 25 nt and 29 nt bands were extracted. For 40 and 20 ng samples, fluorescent marker oligonucleotides were spiked into each sample and a final 15% TBE-Urea polyacrylamide gel was run as described above. Only the 25 nt and 29 nt bands were quantified to determine the final yield. To quantify yield for the ultra-low input samples (2 ng, 400 pg, and 40 pg inputs), all experimental and input control samples were brought to 16 μL with nuclease-free water. Subsequently, 2 μL T4 polynucleotide kinase (PNK) buffer, 1 μL T4 PNK (NEB) and 1 μL ATP [γ-32P]-3000Ci/mmol [10 mCi/mL] (Perkin Elmer NEG002A500UC) were added and incubated for 30 minutes at 37° C. After incubation, unincorporated nucleotides were removed with the RNA Clean and Concentrator-5 kit (Zymo Research R1013) according to manufacturer's instructions. RNA was eluted with 14 μL nuclease-free water, mixed with 2× denaturing gel loading dye (Table 2) and denatured for 90 seconds at 80° C. The samples were electrophoresed, then the gel was incubated in nuclease-free water for 5 minutes followed by a 30 minutes incubation in a 30% methanol and 5% glycerol solution. Both incubations were done on a rocking platform at room temperature. After the incubations, the gel was placed between pre-wetted cellophane sheets (Bio-Rad 1651779) and dried for 2 h in a GelAir drying system (Bio-Rad). The dried gel in cellophane was exposed for at least 12 h to a BAS-IP MS phosphor screen (GE 28956475). The phosphor screen was imaged with a Typhoon FLA 9500 (GE Healthcare) using 500 V PMT at 50 μM resolution. The image was visualized and quantified using ImageJ software only the 25 nt band was quantified as described above. All samples were processed in quadruplicate, with the exception of the Ribo-ITP sample with an RNA ladder input of 20 ng (n=3).

### Cell Culture

Human K562 cells obtained from ATCC were grown in RPMI 1640 medium (Gibco) supplemented with 10% fetal bovine serum (Gibco) and 1% Pen-Strep (Gibco) and incubated at 37° C. with 5% CO2 to a density of ˜2.5×105 cells per ml. Cells were regularly tested for Mycoplasma contamination.

### Size Selection of Purified RNA

To demonstrate the size selection capacity of our on-chip approach, an MNase-digested RNA sample from K562 cells was prepared. Briefly, 3 μL MNase (NEB) was added to a clarified K562 lysate from ˜5M cells and digested for 30 minutes at 37° C., followed by RNA extraction with the miRNeasy Micro kit (Qiagen) per manufacturer's instructions. Ribo-ITP inputs contained 100 ng of the digested, purified RNA. Ribo-ITP was performed as described, with modifications to the collection method. Once the fluorescent marker band reached the interface of the 5% and 10% polyacrylamide gels, the current was suspended and RB was replaced with 12 μL of fresh RB. Ribo-ITP continued until the first fluorescent marker reached the edge of the elution well. The 12 μL of RB in the elution well was collected as Fraction 1 (F1). The well was washed twice with RB then refilled with 12 μL RB. Current was applied again until the front edge of the trailing fluorescent marker began to enter the elution well, and the 12 μL RB elution was collected as Fraction 2 (F2). The elution well was refilled with 12 μL RB and Ribo-ITP was continued for 2 minutes. The final 12 μL elution was collected as Fraction 3 (F3). Control inputs were prepared with the same amounts of bulk RNA and fluorescent markers, then brought to 12 μL with RB. Gel electrophoresis, imaging, and quantification were performed as described.

### Ribosome Profiling Sample Preparation and Monosome Isolation

Approximately 10M K562 cells were pelleted, washed twice with PBS, and immediately flash-frozen in liquid nitrogen. Cells were lysed in 400 μL of cold lysis buffer (Table 2) for 10 minutes on ice and pipetted to homogenize. The lysates were clarified by centrifugation at 1,300×g for 10 minutes at 4° C. Clarified supernatants were digested with 5 μL MNase (NEB M0247S) and incubated for 30 minutes at 37° C. Digestions were stopped with 20 mM ribonucleoside vanadyl complex (NEB: S1402S). The samples were then loaded onto 20%-50% sucrose gradients and ultracentrifuged in a SW41 Ti swinging-bucket rotor (Beckman 331362) at 38,000 rpm for 2.5 h at 4° C. The samples were fractionated using a Biocomp gradient fractionator. RNA was extracted from the monosome fractions with the miRNeasy Micro kit (Qiagen). One third of the eluate was electrophoresed through a 15% TBE-Urea polyacrylamide gel. The ribosome footprints of ˜ 17-35 nt were gel extracted using the crush-and-soak method as described. Final sample resuspension after ethanol precipitation was in 18 μL of nuclease-free water. The purified RNA was dephosphorylated with 1 μL of T4 polynucleotide kinase (NEB) in 1× T4 PNK buffer for 1 h at 37° C. Dephosphorylated ribosome footprints were then ethanol precipitated (300 mM Sodium acetate, 2.5 volumes of ethanol, and 1.5 μL of GlycoBlue) overnight at −20° C. Precipitated RNA was eluted in 10 μL nuclease-free water. The RNA was normalized to 350 ng in 6 μL of nuclease-free water before library preparation.

For 100-cell ribosome profiling experiments, K562 cells were pelleted, washed twice with PBS, and diluted to 100 cells in 5 μL of cold lysis buffer containing cycloheximide. MNase stock (2,000 gel units/μL, NEB) was diluted 1:50 and 1 μL of the dilution was added to the samples. Digestion was performed for 30 minutes at 37° C. in a thermal cycler with a heated lid. 1 μL EGTA was added to a final concentration of 10 mM in order to inhibit further digestion. Samples were placed on ice until processing through Ribo-ITP.

### Mouse Oocyte Isolation

All experiments using mice by the Mouse Genetic Engineering Facility were approved by the Institutional Animal Care and Use Committee at the University of Texas at Austin. Oocytes were collected from superovulated C57BL/6J female mice as previously described (Deng et al., 2014). One hour after human Chorionic Gonadotropin (hCG) injection, the ovaries were placed in a 3 cm dish containing FHM medium (Cytospring, F1114), and Germinal vesicle (GV)-stage oocytes were released by scraping the surface of the ovaries with #5 Dumont forceps (Roboz). Meiosis II (MII)-stage oocytes were isolated from the oviducts approximately 14 h after hCG injection. Cumulus cells were removed from the oocytes by treatment with 1 mg/ml Hyaluronidase (Sigma H3884) in FHM medium. Both GV- and MII-stage oocytes were rinsed through three drops of FHM medium and then through three drops of 20 mg/mL BSA (Sigma A3311) in PBS (Hyclone SH30028.02). The oocytes were placed individually in 0.2 mL PCR tubes using a finely pulled glass pipette under a stereomicroscope and flash-frozen in liquid nitrogen. The liquid volume transferred with the oocytes was less than 0.5 μL.

### In Vitro Fertilization (IVF) Using CAST/EiJ Sperm

Sperm was frozen from CAST/EiJ male mice as described (Love et al., 2014) and stored in liquid nitrogen. For in vitro fertilization, oocytes were isolated from C57BL/6J female mice approximately 15 h after hCG injection, and IVF was performed using thawed CAST/EiJ sperm (Takeo & Nakagata, 2010). 1-cell, 2-cell, 4-cell, and 8-cell embryos were collected 21.5, 39, 62, and 69 h after hCG injection, respectively. Fertilized oocytes were cultured overnight to the 2-cell stage in a 150 μL drop of HTF medium (Cytospring, mH0113). For development to the 4-cell and 8-cell stages, 2-cell embryos were cultured in KSOM medium (Cytospring, K0114). Embryos were placed individually into 0.2 mL PCR tubes and flash-frozen in liquid nitrogen. All samples were processed with Ribo-ITP within 48 h of collection.

A working lysis buffer solution was prepared by adding 1 μL of the MNase (NEB) [1:50 dilution] per 5 μL lysis buffer. To lyse the mouse samples, 6 μL of working lysis buffer was added directly to the frozen cell-containing droplet. Digestion was immediately performed for 30 minutes at 37° C. in a thermal cycler with a heated lid. 1 μL EGTA was added to a final concentration of 10 mM to inhibit further digestion. Samples were placed on ice until processing through Ribo-ITP.

### Ribosome Profiling Library Preparation and Sequencing

Conventional ribosome footprint libraries following monosome isolation (i.e. 350 ng RNA samples in 6 μL nuclease-free water) were generated using the Clontech SMARTer smRNA-Seq kit using 8 PCR cycles (Takara Bio). 30 μL of the PCR reaction was purified with AMPure XP beads (Beckman Coulter A63880) according to the manufacturer's instructions and eluted with 30 μL of nuclease-free water. The final size selection was performed with the BluePippin system (Sage Science) using 3% dye-free agarose cassettes (Sage Science BDQ3010).

For 100-cell human K562 cells and all mouse samples, Ribo-ITP outputs were immediately processed through the D-Plex Small RNA-seq Kit (Diagenode C05030001) with minor modifications as detailed here. The dephosphorylation reaction was supplemented with 0.5 μL T4 PNK (NEB) and the reaction was incubated for 25 minutes. For reverse transcription, the template switching oligo (TSO) was diluted 1:2 in nuclease-free water. All 100-cell human samples and three of the MII-stage oocytes were processed using the single index (SI) module; while the other mouse samples were processed using the unique dual index (UDI) module. Half of the cDNA was amplified for 17 PCR cycles and a 1:4 dilution of the resulting library was assessed by Agilent Bioanalyzer High Sensitivity DNA kit. The concentrations of the target peaks were used to pool samples with approximately equimolar representation. AMPure XP bead cleanup (1.8×) was performed followed by size-selection using 3% agarose, dye-free gel cassettes with internal standards (Sage Science BDQ3010) on the BluePippin platform. Tight parameter settings of 173-207 bp range were used for samples prepared with the SDI module. Tight parameter settings of 183-217 bp range were used for samples prepared with the UDI module. Samples were sequenced on an Illumina NovaSeq 6000.

### Single Cell and Single Embryo RNA Sequencing (RNA-Seq)

Total RNA sequencing libraries were prepared with Smart-seq3 V.3 (Takeo & Nakagata, 2011), with modifications. Unfertilized mouse samples (GV, MII) and in vitro fertilized mouse samples (1, 2, 4, and 8-cell stage) were lysed and reverse transcribed as described. cDNA was pre-amplified with 13 PCR cycles and bead purified with AMPure XP (1.8×) with a final elution in 5 μL nuclease-free water. 1 μL of pre-amplified cDNA was assessed by Bioanalyzer High Sensitivity DNA kit to confirm successful pre-amplification and proper size profile. Another 1 μL was assessed on Qubit using the dsDNA HS assay to quantify the pre-amplified cDNA. Samples were diluted with nuclease-free water and normalized to 600 pg inputs [100 pg/μL] and subjected to tagmentation and post-tagmentation PCR. The tagmentation and subsequent PCR were scaled up 6×: precisely, 600 pg pre-amplified cDNA was tagmented with 6 μL of Tagmentation Mix, 9 μL of Nextera Index primers were added, and 18 μL of Tagmentation PCR mix was used. 16 PCR cycles were performed followed by equivolume sample pooling (12 μL of each PCR product) and AMPureXP purification at a 1× ratio. The final library size distribution and concentration was assessed with the HS DNA Bioanalyzer. Sequencing was performed with Nova Seq 6000 with paired-end reads (using 100 cycle kits: 60+40).

### Computational Processing of Ribosome Profiling Data

Ribosome profiling data were processed using RiboFlow (Hagemann et al.). The first 12 nucleotides from the 5′ end of the reads were extracted using UMI-tools (Ozadam et al., 2020) version 1.1.1 with the following parameters: “umi_tools extract -p “\hat{ }(?P<umi_1>. {12}) (?P<discard_1>.{4}).+$” --extract-method=regex”. The four nucleotides downstream of the UMIs are discarded as they are incorporated during the reverse transcription step. Conventional ribosome profiling samples did not include UMIs.

Next, 3′ adapter AAAAAAAAAACAAAAAAAAAA (SEQ ID NO: 1), was clipped from the Ribo-ITP data, using cutadapt (Smith et al., 2017) version 1.18 with the parameters “-a AAAAAAAAAACAAAAAAAAAA (SEQ ID NO: 1) --overlap=4 --trimmed-only”. For conventional ribosome profiling data, the poly-A tails and the first three nucleotides of the reads were removed using “cutadapt -u 3-a AAAAAAAAAA (SEQ ID NO: 2) --overlap=4 --trimmed-only”.

After UMI extraction and adapter trimming, reads were aligned to ribosomal and transfer RNAs using Bowtie2 (Martin et al., 2011) version 2.3.4.3. The unaligned reads were mapped to a manually-curated transcriptome. Alignments with mapping quality greater than 2 were retained followed by deduplication using UMI-tools when applicable. In deduplication of external libraries without UMIs, a set of reads with the same length that were mapped to an identical nucleotide position were collapsed into a single read. As the last step, .ribo files were created using RiboPy (Hagemann et al.) version 0.0.1. All subsequent analyses used ribosome footprints that were 29-35 nucleotides in length.

For analyses involving nucleotide resolution data, the A-site offset was determined for each ribosome footprint length using translation stop site metagene plots. Specifically, for each read length, the highest peak upstream of the translation stop site was identified and the distance to the annotated stop site was used as the offset.

### Computational Processing of RNA Sequencing Data

5′ adapter sequence “ATTGCGCAATG” (SEQ ID NO: 3) was clipped from the first read in the pair using cutadapt (Smith et al., 2017) version 1.18. Clipped reads shorter than 8 nucleotides were removed using:

“cutadapt-j 4 --trimmed-only-m 8-g ATTGCGCAATG” (SEQ ID NO: 3). The next 8 nucleotides corresponding to the UMIs from the first read in the pair were extracted and appended to the headers (of FASTQ files) of both read pairs using UMI-tools with the following parameters:

“umi_tools extract --bc-pattern NNNNNNNN”. After UMI extraction, the second read in the pair (40 nt) was used for all subsequent analyses.

After filtering out reads aligning to a reference of ribosomal and tRNAs, the remaining reads were aligned to a transcriptome reference where SNPs were masked with Ns (see the next section for details); thereafter, only the alignments with mapping quality greater than 2 were retained. Reads that aligned to the same transcript were collapsed using their respective UMIs: “umi_tools dedup --per-contig --per-gene”. For each transcript, the number of reads aligning to the coding sequence were counted. All alignments used bowtie2 (Martin, 2011) and BAM files were processed using samtools (Langmead & Salzberg, 2012).

Comparison with Polysome Profiling

The transcripts with validated changes in polysomal association between GV- and MII-stage oocytes were obtained from Supplemental FIGS. S2 and S3 of Chen et al. (2011). Of the 29 genes with qrt-PCR validated changes in polysomal association, 28 had the reported direction of effect when comparing the mean of the centered log-ratio (CLR) (Damecek et al., 2021) across the replicates. Specifically, Let M be the geometric mean of all the genes with non-zero counts and let g be the raw counts for a specific gene. Then, CLR of g is computed as clr (g)=In (g/M).

### Allele-Specific Ribosome Occupancy and RNA Expression Analysis

A list of strain-specific single nucleotide polymorphisms (SNPs) was obtained in VCF format from github.com/sandberg-lab/Smart-seq3/blob/master/allele_level_expression/CAST.SNPs.validated.vcf.gz_(Takeo & Nakagata, 2011). 210,004 distinct SNPs were extracted that overlapped with transcript annotations. To avoid alignment biases, transcriptome reference sequences were modified by masking SNP positions with Ns. Mouse sequencing data were aligned to this masked transcriptome reference. For allele-specific analyses reported in FIG. 16A, the 85,339 SNPs within the coding sequences of transcripts were considered. Given that transcripts in oocytes should solely contain maternal SNPs, the data from the MII-stage oocytes was used to construct a simple error correction model. Specifically, 2.67% and 0.40% of reads contained non-maternal sequences in ribosome profiling and RNA-Seq experiments, respectively. These values were used as estimates of the sequencing error percentage (error).

The paternal ratio was defined as (#reads from paternal alleles)/(#reads from paternal alleles+#reads from maternal alleles). For 1-cell to 8-cell embryos, the error-corrected paternal ratio, paternalcorrected, was then calculated as:

$${paternal}_{corrected} = ( 300 \times {paternal}_{observed} - error \times 100 ) / ( 300 - 4 \times error )$$

where paternalobserved is the uncorrected percentage. This equation was derived from the model below under the assumption that sequencing errors were random:

$${paternal}_{observed} = ( {paternal}_{corrected} \times ( 100 - error ) / 100 ) + ( 100 - {paternal}_{corrected} ) \times ( 100 - error / 3 \times 100 ) .$$

For each embryonic stage, to identify the transcripts whose paternal ratios are significantly different in ribosome profiling compared to RNA-Seq, SNP-containing reads for each transcript across replicates were aggregated. Transcripts with more than 10 reads in both ribosome profiling and RNA-seq experiments including at least three maternal and paternal reads were retained. A two-sample test for the equality of proportions with continuity correction (prop.test in R; see Chapter 3 of Fleiss, 2003 for details) was used.

Transcripts with 95% confidence intervals, of difference in paternal ratios (derived from the test for the equality of proportions), overlapping with the interval [−0.05, 0.05] were filtered out. After the p-values were adjusted using the false discovery rate method, transcripts with adjusted p-values less than 0.2 were retained. Transcripts with paternal reads in the MII-stage were also removed, as these likely indicate positions that are prone to alignment errors. As the final step, bootstrapping was applied to establish robustness of the conclusions. Specifically, replicates with replacement were randomly sampled and the statistical testing procedure described above was repeated. 24 transcripts with a false discovery rate less than 0.2 in at least 66 out of 100 bootstrap samples were deemed as having differential allelic ratios.

### Differential Expression and Translation Efficiency Analysis

Reads that align to coding regions were extracted for all experiments. Transcripts with the most variable ribosome occupancy across the developmental stages were determined using the

“FindVariableFeatures” function in the Seurat package v4 with default parameters after read count normalization using a centered log ratio transformation (Fleiss, 2003).

For every pair of consecutive developmental stages, differential RNA expression and translation efficiency was determined using DESeq2 (Berriz et al., 2009). For differential translation efficiency calculation, the interaction term between the developmental stage and measurement modality

(ribosome profiling or RNA-Seq) was used. Default parameters were used for read count normalization and estimation of gene-specific dispersion. Effect size moderation was carried out using the approximate posterior estimation for a generalized linear model (Quinn et al., 2018). The adjusted p-value cutoff was set to 0.01 to determine a set of transcripts with significant changes in RNA expression and translation efficiency. Gene set enrichment analyses for gene ontology terms were carried out using FuncAssociate (http://llama.mshri.on.ca/funcassociate/) with default settings (Cameron & Uhlenbeck, 1977). See Table S2.

TABLE S2
Differential Expression and Translation Efficiency

| | positive fold change | Negative Fold Change |
|---|---|---|
| GV to MII | Enrichment in GV | Enrichment in MII |
| MII to 1 Cell | Enrichment in MII | Enrichment in 1 Cell |
| 2 Cell to 1 Cell | 1 Cell | 2 Cell |
| 4 Cell to 2 Cell | 2 Cell | 4 Cell |
| 8 Cell to 4 Cell | 4 Cell | 8 Cell |

TE Translation Efficiency
RNA Gene expression coming from RNA-Seq

Proteomics Data and Comparison with RNA-Seq and Ribosome Profiling

TMT-labeling based proteomics abundance data for 1-cell to morula stage embryos was obtained from Gao et al. (2017). 3287 proteins had measurements in all three modalities and were used in further analysis. Ribosome occupancy and RNA expression were converted to read density by dividing the read counts by the length of the coding region of each transcript. These values were normalized using a centered log ratio transformation as implemented in Seurat v4 (Fleis, 2003). The similarity between RNA expression, ribosome occupancy and protein abundance was measured using rank correlation with Spearman's correction (Hao et al., 2021; Zhu et al., 2019). The measurement reliability for each modality was estimated using replicate to replicate correlation coefficients (0.71 for ribosome profiling, 0.79 for RNA-seq and 0.8 for mass spectrometry (Gao et al., 2017)).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Abdelmoez et al., Genome Biol., 19:66, 2018.
Abdel-Sayed et al., Anal. Chem., 89:9643-9648, 2017.
Berriz et al., Bioinformatics, 25:3043-3044, 2009.
Bickel et al., Adv. DrugDeliv. Rev., 46:247-279, 2001.
Buenrostro et al., Nature, 523:486-490, 2015.
Cameron & Uhlenbeck, Biochemistry, 16:5120-5126, 1977.
Chen et al., Genes Dev., 25:755-766, 2011.
Danecek et al., Gigascience, 10, 2021.
Deng et al., Science, 343:193-196, 2014.
Eid & Santiago, Lab Chip, 18:11-26, 2017.
Evsikov et al., Mol. Reprod. Dev., 76:805-818, 2009.
Fleiss, Statistical Methods for Rates and Proportions, J. Wiley, 2003.
Gao et al., Cell Rep., 21:3957-3969, 2017.
Gebauer et al., EMBO J., 13:5712-5720, 1994.
Gerashchenko et al., Nucleic Acids Res., 2016.
Green & Sambrook, Cold Spring Harb. Protoc., 2019, 2019.
Hafemeister & Satija, Genome Biol., 20:296, 2019.
Hagemann et al., protocols.io, Smart-seq3 Protocol v3
Han et al., Lab Chip, 19:2741-2749, 2019.
Handbook of Pharmaceutical Salts: Properties, and Use, Stahl and Wermuth Eds.), Verlag Helvetica Chimica Acta, 2002.
Hao et al., Cell, 184:3573-3587, 2021.
Ingolia et al., Science, 324:218-223, 2009.
Ingolia et al., Cold Springs Harb. Perspect. Biol., 11, 2019.
Khnouf et al., Anal. Chem., 90:12609-12615, 2018.
Khnouf et al., Electrophoresis, 40:3140-3147, 2019.
Kuriyama et al., Electrophoresis, 36:1658-1662, 2015.
Langmead & Salzberg, Nat. Methods, 9:357-359, 2012.
Li et al., Development, 137:859-870, 2010.
Love et al., Genome Biol., 15:550, 2014.
March's Advanced Organic Chemistry: Reactions, Mechanisms, and Structure, 2007.
Marshall et al., J. Chromatogr. A., 1331:139-142, 2014.
Martin et al., EMBnet.journal, 17:10-12, 2011.
McGlincy & Ingolia, Methods, 126:112-129, 2017.
Nagaoka et al., Nat. Rev. Genet., 13:493-504, 2012.
Oh et al., Development, 127:3795-3803, 2000.
Ozadam et al., Bioinformatics, 36:2929-2931, 2020.
Paris et al., Cell Stem Cell, 25:137-148, 2019.
Quinn et al., Bioinformatics, 34:2870-2878, 2018.
Reid et al., Methods, 91:69-74, 2015.
Rogacs et al., J. Chromatogr. A., 1335:105-120, 2014.
Schier et al., Nat. Methods, 17:17-20, 2020.
Schoch et al., Lab Chip, 9:2145-2152, 2009.
Smith et al., Genome Res., 27:491-499, 2017.
Szollosi et al., J. Cell. Sci., 11:521-541, 1972.
Takeo & Nakagata, Biol. Reprod., 85:1066-1072, 2011.
Takeo & Nakagata, Lab. Anim., 44:132-137, 2010.
Tang et al., Nat. Methods, 6:377-382, 2009.
VanInsberghe et al., Nature, 597:561-565, 2021.
Vastenhouw et al., Development, 146, 2019.
Vu et al., Nat. Med., 23:1369-1376, 2017.
Wang et al., Biol. Reprod., 65:748-754, 2001.
Wolin & Walter, EMBO J., 7:3559-3569, 1988.
Zhao et al., Bioinformatics, 35:1486-1493, 2019.
Zhu et al., Bioinformatics, 35:2084-2092, 2019.

What is claimed is:

1. A method for obtaining one or more nucleic acids comprising:
(A) obtaining a purified cell lysate containing one or more nucleic acid comprising less than 100 pg of the one or more nucleic acids; and
(B) separating the purified cell lysate in a size selection channel to detect one or more nucleic acids.

2. The method of claim 1, wherein the one or more nucleic acids are ribosome protected fragments (RPFs).

3. The method of either claim 1 or claim 2, wherein the RPFs are ribonucleic acids.

4. The method of claim 1, wherein the one or more nucleic acids are siRNA.

5. The method of claim 1, wherein the one or more nucleic acids are microRNA.

6. The method of claim 1, wherein the one or more nucleic acids are the degradation products of a cell.

7. The method according to any one of claims 1-6, wherein the one or more nucleic acids comprise from about 10 nucleotides to about 50 nucleotides.

8. The method according to any one of claims 1-7, wherein the one or more nucleic acids comprise from about 15 nucleotides to about 40 nucleotides.

9. The method according to any one of claims 1-8, wherein the one or more nucleic acids comprise from about 17 to about 35 nucleotides.

10. The method according to any one of claims 1-9 wherein the method further comprises digesting the obtained purified cell lysate with a nuclease.

11. The method of claim 10, wherein the method further comprises terminating the digestion with a nuclease inhibitor.

12. The method of claim 10, wherein the method further comprises terminating the digestion with a chelator.

13. The method according to any one of claims 1-12, wherein the method further comprises pretreating the size selection channel.

14. The method according to any one of claims 1-12, wherein the pretreating comprises pretreating with one or more solutions.

15. The method of claim 14, wherein the pretreating comprises treating with a cross-linking solution.

16. The method of claim 15, wherein the cross-linking solution comprises benzophenone.

17. The method of claim 16, wherein the cross-linking solution comprises from about 1% w/v to about 20% w/v of the benzophenone.

18. The method of claim 17, wherein the cross-linking solution comprises about 10% w/v benzophenone.

19. The method according to any one of claims 1-18, wherein the method further comprises loading the size selection channel.

20. The method of claim 19, wherein the size selection channel comprises one or more discrete separation zones.

21. The method of either claim 19 or claim 20, wherein the size selection channel comprises two or more discrete separation zones.

22. The method according to any one of claims 19-21, wherein the size selection channel comprises a first separation zone and a second separation zone.

23. The method of claim 22, wherein the first separation zone is loaded with a polymer.

24. The method of claim 23, wherein the first separation zone consists essentially of a polymer.

25. The method of either claim 23 or claim 24, wherein the polymer is polyacrylamide.

26. The method according to claim 23-25, wherein the first separation zone is loaded with from about 0.1% to about 20% polyacrylamide.

27. The method according to any one of claims 23-26, wherein the first separation zone is loaded with from about 5% to about 15% polyacrylamide.

28. The method according any one of claims 23-27, wherein the first separation zone is loaded with about 10% polyacrylamide.

29. The method according to any one of claims 20-28, wherein the size selection channel comprises a second separation zone.

30. The method of claim 29, wherein the second separation zone is loaded with a polymer.

31. The method of claim 30, wherein the polymer is polyacrylamide.

32. The method of either claim 30 or 31, wherein the polyacrylamide is between about 0.1% and 20% polyacrylamide.

33. The method according to any one of claims 30-32, wherein the polyacrylamide is between about 1% and 10% polyacrylamide.

34. The method according to any one of claims 30-33, wherein the polyacrylamide is about 5% polyacrylamide.

35. The method according to any one of claims 19-34, wherein the polymer is impregnated with an initiator.

36. The method according to claim 35, wherein the initiator is photoactivatable.

37. The method of claim 36, wherein the initiator is 2,2′-azobis [2-methyl-N-(2-hydroxyethyl) propionamide].

38. The method according to any one of claims 1-37, wherein the method further comprises initiating the polymerization of the polymer by exposing one or more acrylamide monomers to light.

39. The method of claim 38, wherein the light is 200 nm to about 800 nm.

40. The method of either claim 38 or claim 39, wherein the light is from about 300 nm to about 600 nm.

41. The method according to any one of claims 38-40, wherein the light is 365 nm.

42. The method according to any one of claims 1-41, wherein the size selection channel comprises a thickness from about 100 μm to about 500 μm.

43. The method according to any one of claims 1-42, wherein the size selection channel comprises a thickness of about 375 μm.

44. The method according to any one of claims 1-43, wherein the method further comprises adding at least one labeling agent to the purified cell lysate.

45. The method according to claim 44, wherein at least one of the labeling agents is a nucleic acid.

46. The method of either claim 44 or claim 45, wherein the labeling agent is DNA, RNA, or dideoxyribonucleic acid.

47. The method according to any one of claims 44-46, wherein at least one of the nucleic acids is a dideoxyribonucleic acid.

48. The method according to either claim 44 or 45, wherein at least one of the nucleic acids is a ribonucleic acid.

49. The method according to any one of claims 44-48, wherein at least one of the labeling agents is unable to be amplified.

50. The method of claim 49 wherein at least one of the labeling agents is a 3′-dideoxynucleoside.

51. The method of claim 49, wherein at least one of the labeling agents is a 3′-deoxynucleoside.

52. The method according to any one of claims 44-51, wherein at least one of the labeling agents is able to be detected.

53. The method according to any one of claims 44-52, wherein the method comprises monitoring the movement of the labeling agents.

54. The method of either claim 52 or claim 53, wherein at least one of the labeling agents comprises a fluorescent dye.

55. The method of claim 54, wherein the fluorescent dye comprises an emission spectrum from about 300 nm to about 900 nm.

56. The method of claim 55, wherein the emission spectrum is from about 400 nm to about 700 nm.

57. The method according to any one of claims 54-56, wherein the fluorescent dye is an ATTO dye, an Alexa Fluor dye, a rhodamine dye, or a fluorescein dye.

58. The method according to any one of claims 54-57, wherein the fluorescent dye is an ATTO dye.

59. The method according to any one of claims 44-58, wherein the labeling agent migrates through the size selection channel at a rate approximately equivalent to an oligomer from about 5 deoxyribonucleotides to about 35 deoxyribonucleotides.

60. The method of claim 59, wherein the oligomer is from about 10 deoxyribonucleotides to about 35 deoxyribonucleotides.

61. The method of claim 60, wherein the oligomer is from about 12 deoxyribonucleotides to about 32 deoxyribonucleotides.

62. The method of claim 61, wherein the oligomer is from about 15 deoxyribonucleotides to about 25 deoxyribonucleotides.

63. The method of claim 62, wherein the oligomer is about 19 deoxyribonucleotides.

64. The method according to any one of claims 44-63, wherein the labeling agent migrates through the size selection channel at a rate corresponding to an oligomer from about 5 deoxyribonucleotides to about 35 deoxyribonucleotides.

65. The method of claim 64, wherein the oligomer is from about 20 deoxyribonucleotides to about 75 deoxyribonucleotides.

66. The method of claim 65, wherein the oligomer is from about 25 deoxyribonucleotides to about 60 deoxyribonucleotides.

67. The method of claim 66, wherein the oligomer is from about 30 deoxyribonucleotides to about 45 deoxyribonucleotides.

68. The method of claim 67, wherein the oligomer is about 36 deoxyribonucleotides

69. The method according to any one of claims 44-68, wherein the method comprises adding two labeling agents to the purified cell lysate.

70. The method of claim 69, wherein the two labeling agents comprise a labeling agent that migrates through the size selection channel at a rate approximately equivalent to an oligomer of 19 deoxyribonucleotides.

71. The method of either claim 69 or claim 70, wherein the two labeling agents comprise a labeling agent that migrates through the size selection channel at a rate approximately equivalent to an oligomer of 36 deoxyribonucleotides.

72. The method according to any one of claims 69-71, wherein the two labeling agents are a labeling agents that migrates through the size selection channel at a rate approximately equivalent to an oligomer of 19 and 36 deoxyribonucleotides.

73. The method according to any one of claims 1-72, wherein the purified cell lysate is derived from a sample of about 1 cell to about 1 million cells.

74. The method of claim 73, wherein the purified cell lysate is derived from a sample of about 1 cell to about 100,000 cells.

75. The method of claim 74, wherein the purified cell lysate is derived from a sample of about 1 cell to about 100 cells.

76. The method of claim 75, wherein the purified cell lysate is derived from a sample of about 1 cell.

77. The method according to any one of claims 1-76, wherein the purified cell lysate is of a mammalian cell population.

78. The method of claim 77, wherein the mammalian cell population is a human cell population.

79. The method of claim 78, wherein the human cell is an embryonic cell population.

80. The method of claim 78, wherein the human cell is a FACS sorted human cell population.

81. The method of claim 78, wherein the human cell is an immune cell population.

82. The method of claim 81, wherein the immune cell population is a population of B cells.

83. The method of claim 81, wherein the immune cell population is a population of T cells.

84. The method of claim 78, wherein the human cell is a cancer cell population.

85. The method of claim 84, wherein the cancer cell population is a population of cancer stem cells.

86. The method according to any one of claims 1-85, wherein the mass of the one or more nucleic acids in the purified cell lysate is less than 80 picograms.

87. The method of claim 86, wherein the mass is less than 60 picograms.

88. The method of claim 87, wherein the mass is less than 40 picograms.

89. The method according to any one of claims 1-88, wherein the mass of the one or more nucleic acids in the purified cell lysate is from about 1 picograms to about 100 picograms.

90. The method according to any one of claims 1-89, wherein the mass of the one or more nucleic acids in the purified cell lysate is about 10 picograms to about 80 picograms.

91. The method according to any one of claims 1-90, wherein the mass of the one or more nucleic acids in the purified cell lysate is about 40 picograms.

92. The method according to any one of claims 1-91 wherein the separation of the purified cell lysate is by isotachophoresis.

93. The method of claim 92, wherein the isotachophoresis comprises applying a current across the size selection channel.

94. The method of claim 93, wherein the current is a constant current.

95. The method of either claim 93 or claim 94, wherein the isotachophoresis comprises applying a voltage across the size selection channel.

96. The method according to any one of claims 92-95, wherein the separation comprises applying the purified cell lysate in a buffer solution.

97. The method of claim 96, wherein the buffer solution comprises a buffering agent.

98. The method of claim 97, wherein the buffering agent is tris or bis-tris.

99. The method of either claim 97 or claim 98, wherein the buffering agent is bis-tris.

100. The method according to any one of claims 96-99, wherein the buffer solution further comprises a surfactant.

101. The method according to any one of claims 96-100, wherein the buffer solution further comprises a fungicide.

102. The method according to any one of claims 96-101, wherein buffer solution further comprises a reducing agent.

103. The method according to any one of claims 96-102, wherein the buffer solution comprises one or more salts.

104. The method according to any one of claims 96-103, wherein the separation further comprises adding an electrolyte solution.

105. The method of claim 104, wherein the electrolyte solution further comprises a buffer.

106. The method of either claim 104 or claim 105, wherein the method comprises using a first electrolyte solution and a second electrolyte solution.

107. The method according to any one of claims 104-106, wherein the first electrolyte solution further comprises an acid.

108. The method according to any one of claims 104-107, wherein the second electrolyte solution comprise a second buffer.

109. The method according to any one of claims 92-108, wherein the separation comprises applying a positive and negative electrode to the size separation channel.

110. The method of claim 109, wherein the positive and negative electrodes are applied to separate ends of the size separation channel.

111. The method of claim 110, wherein the positive and negative electrodes are applied at opposite ends of the size separation channel.

112. The method according to any one of claims 92-111, wherein the first electrolyte solution is applied to the same end of the size separation channel as the positive electrode.

113. The method according to any one of claims 92-112, wherein the second electrolyte solution is applied to the same end of the size separation channel as the negative electrode.

114. The method according to any one of claims 1-113, comprising stopping the application of current when the longer labeling agent enters the second separation zone.

115. The method of claim 114 further comprising emptying a collection well while the current is stopped.

116. The method of either claim 114 or claim 115, wherein the current is restarted after the well has been emptied.

117. The method according to any one of claims 114-116, wherein the current is applied until the shorter labeling agent enters a collection well.

118. The method according to any one of 1-222, wherein the method comprises collecting the one or more nucleic acids in a collection well.

119. The method of either claim 117 or claim 118, wherein the collection well has been filled with a dephosphorylation buffer.

120. The method of claim 119, wherein the dephosphorylation buffer further comprises a buffering agent.

121. The method of either claim 119 or claim 120, wherein the dephosphorylation buffer further comprises one or more salts.

122. The method according to any one of claims 119-121, wherein the dephosphorylation buffer further comprises a reducing agent.

123. The method according to any one of claims 1-122, wherein the method further comprises sequencing the one or more nucleic acids.

124. The method according to any one of claims 1-123, wherein the method further comprises quantifying the one or more nucleic acids.

125. A method of obtaining one or more ribosome protected fragments (RPFs) comprising:
(A) obtaining a purified cell lysate containing one or more nucleic acid;
(B) separating the purified cell lysate in a size selection channel to obtain one or more separated ribosome protected fragments.

126. The method of claim 125, wherein the purified cell lysate comprises less than 100 pg of nucleic acid.

127. A method of quantifying one or more ribosome protected fragments (RPFs) comprising:
(A) obtaining a purified cell lysate containing one or more nucleic acid;
(B) separating the purified cell lysate in a size selection channel to obtain one or more separated ribosome protected fragments; and
(C) quantifying the separated ribosome protected fragments.

128. A method of determining the sequence of one or more ribosome protected fragments (RPFs) comprising:
(A) obtaining a purified cell lysate containing one or more nucleic acid;
(B) separating the purified cell lysate in a size selection channel to obtain one or more separated ribosome protected fragments; and
(C) sequencing the separated ribosome protected fragments to determine the sequence of the ribosome protected fragments.

129. An apparatus for detecting one or more ribosome protected fragments (RPFs), the apparatus comprising:
a reservoir containing a first electrolyte solution;
a reservoir containing a second electrolyte solution; and
a channel, wherein:
the channel extends between the reservoir containing the first electrolyte solution and the reservoir containing the second electrolyte solution; and
the channel contains a liquid and a polyacrylamide gel.

130. The apparatus of claim 129 further comprising an elution well.

131. The apparatus of any one of claims 129-130 wherein the first electrolyte solution is a leading electrolyte solution and the second electrolyte solution is a trailing electrolyte solution.

132. The apparatus of any one of claims 129-131 wherein the polyacrylamide gel varies in concentration in the liquid between the reservoir containing the first electrolyte solution and the reservoir containing the second electrolyte solution.

133. The apparatus of any one of claims 129-132 wherein the apparatus comprises a plurality of reservoirs containing the first electrolyte solution.

134. The apparatus of claim 133 wherein:
the plurality of reservoirs containing the first electrolyte solution comprises a first reservoir, a second reservoir and a third reservoir;
the channel contains a cell lysate between the reservoir containing the second electrolyte solution and the first reservoir containing the first electrolyte solution;
the channel contains a first concentration of polyacrylamide gel between the first reservoir containing the first electrolyte solution and the second reservoir containing the first electrolyte solution;
the channel contains a second concentration of polyacrylamide gel between the second reservoir containing the first electrolyte solution and the third reservoir containing the first electrolyte solution; and
the second concentration of polyacrylamide gel is greater than the first concentration of polyacrylamide gel.

135. The apparatus of claim 134 wherein the first concentration of polyacrylamide gel is approximately 5 percent and the second concentration of polyacrylamide gel is approximately 10 percent.

136. The apparatus of claim 134 or 135 wherein the apparatus comprises an elution well proximal between the second reservoir containing the first electrolyte solution and the third reservoir containing the first electrolyte solution.

137. The apparatus of any one of claims 129-136, further comprising:
a power supply coupled to a first electrode and a second electrode, wherein:
the first electrode is located in the reservoir containing the first electrolyte solution; and
the second electrode is located in the reservoir containing the second electrolyte solution.

138. The apparatus of any one of claims 129-137, further comprising a control circuit configured to control the power supply.

139. The apparatus of any one of claims 129-138 wherein the control circuit is configured to control the power supply to apply approximately 300 milliamperes (mA) to the channel.

140. The apparatus of any one of claims 129-139 wherein the channel has a thickness of approximately 375 μm.

141. The apparatus of any one of claims 129-140 wherein the channel is formed from polydimethylsiloxane (PDMS).


SEQUENCE LISTING (8 sequences)

SEQ ID NO: 1 - RNA, 21 nt, Synthetic construct
  source 1..21: mol_type = other RNA
aaaaaaaaaacaaaaaaaaaa

SEQ ID NO: 2 - RNA, 10 nt, Synthetic construct
  source 1..10: mol_type = other RNA
aaaaaaaaaa

SEQ ID NO: 3 - RNA, 11 nt, Synthetic construct
  source 1..11: mol_type = other RNA
attgcgcaatg

SEQ ID NO: 4 - DNA, 18 bp, Synthetic construct
  source 1..18: mol_type = other DNA
ctgctggagttcgtgacc

SEQ ID NO: 5 - DNA, 35 bp, Synthetic construct
  source 1..35: mol_type = other DNA
cctccttgtataaatcctggttgctgtctctttat

SEQ ID NO: 6 - RNA, 32 nt, Synthetic construct
  source 1..32: mol_type = other RNA
rartrcrargrgrgrcrcrartrararcrarg

SEQ ID NO: 7 - RNA, 52 nt, Synthetic construct
  source 1..52: mol_type = other RNA
rartrgrtrtrargrgrgrartrararcrargrgrgrtrarartrgrcrgra

SEQ ID NO: 8 - RNA, 68 nt, Synthetic construct
  source 1..68: mol_type = other RNA
rartrgrtrarcrarcrtrargrgrgrartrararcrargrgrgrtrarartrcrararcrgrcrgra